(19) United States
(12) Patent Application Publication
(10) Pub. No.: US 2003/0078515 A1
(43) Pub. Date: Apr. 24, 2003

(54) System and method for remotely calibrating a system for administering interactive hearing tests

(75) Inventors: Christoph Menzel, Madison, CT (US); John H. Winstead, Sunnyvale, CA (US); Terri E. Ives, San Jose, CA (US); Sunil Puria, Mountain View, CA (US); Andy P. Atamaniuk, Redwood City, CA (US); Brent W. Edwards, San Francisco, CA (US)

Correspondence Address: HAYNES BEFFEL & WOLFELD LLP, P O BOX 366, HALF MOON BAY, CA, 94019, US

(73) Assignee: SOUND ID

(21) Appl. No.: 09/975,863

(22) Filed: Oct. 12, 2001

(51) Int. Cl. A61B 5/00

(52) U.S. Cl. 600/559; 600/300

(57) ABSTRACT

A technique allows a web site visitor, or other user of a consumer electronic device that is remote from a hearing test server, to calibrate the device in a self-administered fashion, and to apply the calibration in measuring their hearing loss. The measurements can be applied as a hearing profile having a level of quality which can be used for customizing audio products. Audio resources on consumer electronic device such as a home computer, a hand-held computing platform, a mobile phone, or other device that includes audio resources sufficient to support self-administered hearing tests are calibrated. The method includes prompting a user of the device to make a calibration sound using an item likely to be available to the user, other than audio resources on the device. Next, a calibration test using the audio resources on the device to calibrate the device with reference to the calibration sound is run.

### RELATED APPLICATION DATA

The present application is related to co-pending and commonly owned U.S. patent application Ser. No. 09/830,480, INTERNET BASED HEARING ASSESSMENT METHODS, invented by Menzel et al.; filed Apr. 26, 2001; and to co-pending and commonly owned U.S. patent application Ser. No. ______, SYSTEM AND METHOD FOR REMOTELY ADMINISTERED, INTERACTIVE HEARING TESTS, invented by Edwards, et. al; filed on the same day as the present application.

### BACKGROUND OF THE INVENTION

1. Field of the Invention

The present invention relates to methods and systems for remotely administering hearing tests, in which the subjects of the test use consumer electronic equipment coupled to communication media, such as Internet connected personal computers, cell phones, personal digital assistants, personal audio equipment, and the like, for the generation of stimuli during the test.

2. Description of Related Art

Hearing tests are used to develop hearing profiles of persons, which can be used for fitting hearing aids and for other diagnostic purposes. Professional audiologists are typically required for conducting the tests needed to provide a hearing profile, because of the large number of factors involved in making an assessment necessary for generating a reliable hearing profile. An audiologist is able to set up a controlled environment, and conduct the test according to a testing protocol involving a number of stimuli and response steps that is adapted based on the responses gathered during the test.

The hearing profiles of individuals vary in a number of ways. The ability to hear sounds varies with frequency among individuals across the normal audio frequency range. Also, the dynamic range varies among individuals so that levels of an audio stimulus that are perceived as soft sounds and levels of an audio stimulus that are perceived as loud sounds differ from person to person. Standard hearing tests are designed to produce an audiogram that characterizes such factors as frequency, sensitivity and dynamic range in the hearing profiles of individuals. There are also other factors that affect a hearing profile. For example, psycho-acoustic factors concerning the manner in which a person perceives combinations of normal sounds affect the ability to hear in ways that can vary from person to person. Also, environmental factors such as the usual listening environment of a person (library, conference room, concert hall) and the equipment on which the sound is produced (loud speakers, ear phones, telephone hand set) are important. In persons wearing hearing aids or using other assistive hearing devices, the type of aid or device affects the hearing profile. The physiology of an impairment suffered by the individual may also be an important factor in the hearing profile.

The hearing profiles of individuals have been applied in the hearing aid field for customizing and fitting hearing aids for individuals. See, for example, U.S. Pat. No. 4,731,850 entitled PROGRAMMABLE DIGITAL HEARING AID SYSTEM, invented by Levitt et al.; and U.S. Pat. No. 5,848,171 entitled HEARING AID DEVICE INCORPORATING SIGNAL PROCESSING TECHNIQUES, invented by Stockham, Jr. et al. Thus, techniques for processing sound to offset variations in hearing are well known. However, these techniques are unavailable to persons not using hearing aids. Furthermore, many persons who could benefit from such processing are not in position to use hearing aids for a variety of reasons.

A variety of uses for hearing profiles, other than for the purposes of prescribing hearing aids and assistive listening devices, is being developed. For example, hearing profiles of individuals can be utilized for producing customized audio products, such as pre-recorded music that has been modified according to the hearing profile of the listener. One medium for delivering customized audio products is the Internet. See, co-pending U.S. patent application No. xxxx, entitled SOUND ENHANCEMENT FOR MOBILE PHONES AND OTHER PRODUCTS PRODUCING PERSONALIZED AUDIO FOR USERS, invented by Rader, et al. filed xxxx (RXSD1009-1); and co-pending U.S. patent application Ser. No. 09/464,036, entitled SYSTEM AND METHOD FOR PRODUCING AND STORING HEARING PROFILES AND CUSTOMIZED AUDIO DATA BASED ON SUCH HEARING PROFILES, invented by Pluvinage, et al., filed Dec. 15, 1999.

Because of the difficulty in obtaining a hearing assessment test, and for a variety of other reasons, many persons who could benefit from devices that would assist their hearing do not follow through with obtaining a prescription for such devices. Thus, it is desirable to simplify the procedures involved in obtaining a reliable hearing assessment.

U.S. Pat. No. 5,928,160 describes a home hearing test system and method based on the use of calibrated headphones specially manufactured to support the hearing test using home audio equipment. In addition, reference is made to this patent for its discussion of background concerning hearing assessment tests in general. However, home hearing assessment tests have not achieved commercial acceptance.

Some efforts have been made to develop a technique for allowing a web site visitor to measure their hearing loss in an efficient and consistent way that is self-administered. (See, web sites: “www.handtronix.com,” “www.onlinehearing.com,” “www.nigelworks.com/Pages/software/hearingtest/Version3.5/indexforv3.5.html,” “http://weinstein.cncfamily.com/AAI/,” “www.freehearingtest.com,” and “www.didyouhearme.com.”) Some of these attempts have implemented procedures that are similar to if not identical to a clinical audiogram, where a tone is presented and the listener responds if they heard the sound, in a type of yes-no threshold test. Other attempts implement a screening procedure where tones are presented and results are based on whether or not you heard those tones with no adjustment of sound presentation based on user response.

For all remotely administered tests, calibration of the remote system is problematic. Without reliable calibration, interpretation of the tests results suffers.

As the Internet gains popularity, and more individuals obtain the general-purpose processing power of personal computers coupled to the Internet and having sound cards or other audio processing capability, the Internet is becoming a more important medium for the delivery of audio products. Accordingly, it is desirable to leverage the communication technology the Internet used in the delivery of audio products for the purposes of performing hearing assessments in the home.

### SUMMARY OF THE INVENTION

The present invention provides a technique allowing a web site visitor, or other user of a consumer electronic device that is remote from a hearing test server, to calibrate the device in a self-administered fashion, and to apply the calibration in measuring their hearing loss. The measurements can be applied as a hearing profile having a level of quality which can be used for customizing audio products.

Embodiments of the invention provide a method for calibrating audio resources on consumer electronic device such as a home computer, a hand-held computing platform, a mobile phone, or other device that includes audio resources sufficient to support self-administered hearing tests. The method includes prompting a user of the device to make a calibration sound using an item likely to be available to the user, other than audio resources on the device. Next, the method provides for executing a calibration process using the audio resources on the device to calibrate the device with reference to the calibration sound.

The present invention is based on the realization that the variability in loudness and spectral content of a great many sounds does not vary that much across a group of individuals making the sound when simple instructions are given. Hence, one of these sounds, a calibration sound, can be used as a reference sound. Compelling the computer to produce a masking sound while the calibration sound is produced, the threshold of masking can be found by adjusting the output level of the computer. Now, since the calibration sound level and spectral content is reasonably well known it is understood that the masking level needed at the masking threshold can be known. With the relationship between the calibration sound, the masking sound, and the known level of the calibration sound, the system is considered calibrated.

According to various embodiments, the calibration sound is created using common items likely to be available to the user. If the user is expected to be operating a home computer, then the item is selected that is likely to be present near the computer. In this example, the item used for generating the calibration sound may comprise a key chain, a piece of paper, a coin, the paper clip, a pencil, a pen, the computer keyboard, or other desktop item. If the user is likely to be in an automobile, then the item selected for use in generating the calibration sound would be likely to be present in the car. Thus, items likely to be available to the user are selected based upon the environment in which the user is likely to be executing the process.

In one embodiment, the calibration sound is created using two pieces of paper, where the first piece of paper is laid on the desktop, and the second piece of paper is held in the hand and rubbed on the first piece. The rubbing sound made by this action acts as the calibration sound. The loudness and frequency characteristics of the rubbing sound can be predicted within acceptable ranges, so that a calibration of the electronic device is achieved based on this calibration sound.

A calibration process executed by the electronic device in one embodiment includes a process for determining a setting of the audio resources on the device which results in production of a masking sound that masks, or “drowns out,” the calibration sound. The determination of whether the masking sound masks the calibration sound may be done by the user of the process making a judgement while listening to the sounds, or may be made using electronic sensors like microphones, along with comparison logic.

The setting of a programmable volume control parameter, or other audio parameter, on the device is recorded for the purposes of the calibration. This setting can be used in the generation of stimulus for a self-administered hearing test using the electronic device. For example, in one embodiment, the calibration is used for the generation of stimulus that has a sound pressure level with a high probability of being within about 10 dB of a predicted level, where a high probability in this context is about 90 to 95 percent.

In embodiment in which the electronic device includes the display, the step of prompting the user includes display instructions to the user on the display. Instructions included description of the technique for making the calibration sound using the item, and a description of the process for controlling the device to generate a masking sound.

Thus, the instructions identify the item to be used, an display instructions including a description of a technique for making the calibration sound using the item. Thus, where the item comprises a sheet paper, instructions describe a technique for making the calibration sound by rubbing the sheet of paper. Where the item comprises two sheets of paper, then the instructions included a description of a technique for making the calibration sound by folding a first piece of paper in half (or in quarters or in other configurations), laying a second piece of paper on a desk, and rubbing the first sheet paper on the second sheet paper. Where the item comprises an alpha numeric keyboard, such as the keyboard on a personal computer, the instructions included a description of a technique for making calibration sound by striking the keyboard. Other calibration sounds are made in response to instructions for rubbing a pencil on surface, tapping a desk, dropping a coin on a surface, and jingling a key chain, or other such processes.

In one embodiment, the calibration process comprises an interaction in which the user supplies input signals to adjust the masking sound until the condition is met. In one embodiment, the process includes a routine which automatically adjusts the masking sound, such as by adjusting the volume of the masking sound according to the predefined formula, and conducts an interaction in which the user supplies input signal to indicate that the condition is met. In another embodiment, the user supplies input signals to adjust a masking sound, such as to increase or decrease its volume, until the condition is met.

Embodiments of the invention include a process for determining if the user can hear the calibration sound, and if not then executing an alternative calibration process. In one example, the alternative process includes determining a setting at which the user can hear a set of test sounds. Further, embodiments of the invention are provided in which the process includes at least one flow that is executed if an error condition occurs, that prompts the user to perform an act to correct a possible source of the error.

In various embodiments, the process includes providing instructions to the user describing the calibration process. The instructions in this embodiment instruct the user to signal completion of the process when a condition is achieved in which the masking sound masks calibration sound, and wherein the instructions use terminology semantically equivalent to “drowns out” to describe the condition. It is found that a significantly greater number of users properly understand the calibration process if the terminology “drowns out” or a semantic equivalent of “drowns out” is used in the instructions, than if the terminology used includes “masks,” or “matches.” One semantic equivalent is “washes out.”

In one embodiment, the invention is a method for conducting a hearing test using a computer program. The method includes establishing a communication channel between a remote device and server in a communication network. A first component of the computer program is executed on the server, and a second component of the computer program is executed at the remote device. The computer program according to the invention comprises a routine to manage interaction via an interface on the remote device, and adaptively select stimuli based upon said interaction to be produced at the remote device according to a convergent process to determine a hearing characteristic. The interaction comprises an N-alternative forced choice interaction in one embodiment. The convergent, adaptive process comprises a staircase function or a maximum likelihood function in alternative embodiments of the invention.

According to various embodiments of the present invention, the remote device communicates with the server via a packet switched network, such as the Internet, which may establish links via wired or wireless communication media. Also, the remote device may communicate with the server via a cellular telephone network, a pager network, or any of a variety of communication technologies.

Also, according to various embodiments of present invention, the remote device comprises a mobile phone, a home computer, a hand-held computing platform, or other consumer electronic devices, such as home stereo or television equipment.

The present invention also provides an apparatus that comprises a data processor, a communication interface and memory which stores instructions in a form readable and executable by the data processor. The instructions specify processes which establish a communication channel with a remote device via the communication interface, and manage the calibration and presentation of interaction with the calibration process and hearing test subject via an interface on the remote device. The data processor in this embodiment of the invention acts as a server which manages hearing tests remotely, enabling test subjects to self administer the tests using a variety of consumer electronic devices. In one embodiment, the apparatus comprises routines for downloading software components to the remote device for use during the interaction.

Thus, the present invention enables remote, self-administered hearing tests managed using communication technology such as the Internet and a variety of consumer electronics devices as a test terminal.

Other aspects and advantages of the present invention can be seen on review of the drawings, the detailed description, and the claims which follow.

### BRIEF DESCRIPTION OF THE FIGURES

FIG. 1 illustrates an Internet based system for conducting a hearing assessment test according to the present invention.

FIG. 2 is a flow chart illustrating the method of operation for an Internet based test according to present invention.

FIG. 3 provides a perspective of a variety of consumer electronic devices at which test subjects may take hearing tests according to the present invention.

FIG. 4 illustrates signals generated for a three alternative forced choice step.

FIG. 5 illustrates signals generated for a two alternative forced choice step.

FIG. 6 illustrates a process flow for a convergent staircase procedure used with one embodiment of the present invention.

FIG. 7 illustrates a basic calibration set up used with one embodiment of the present invention.

FIG. 8 is a plot showing mixer levels during the calibration procedure in one embodiment of the present invention.

FIG. 9 is a plot showing management of signal levels during a calibration process in one embodiment of the present.

FIGS. 10A and 10B together provide a flow chart for a testing procedure in one embodiment of the present invention.

FIGS. 11-24 are images of web pages used for presentation of a hearing test according to one embodiment of the present invention.

FIGS. 25A and 25B illustrate a main flow of a calibration process according to the present invention.

FIGS. 26A and 26B illustrate an alternative calibration flow in the event the user cannot hear the calibration sound prompted in the flow of FIGS. 25A and 25B.

FIG. 27 illustrate an error flow arising from the alternative calibration flow of FIGS. 26A and 26B.

FIGS. 28A and 28B illustrate error flows arising from the main flow of the calibration process shown in FIGS. 25A and 25B..

### DETAILED DESCRIPTION

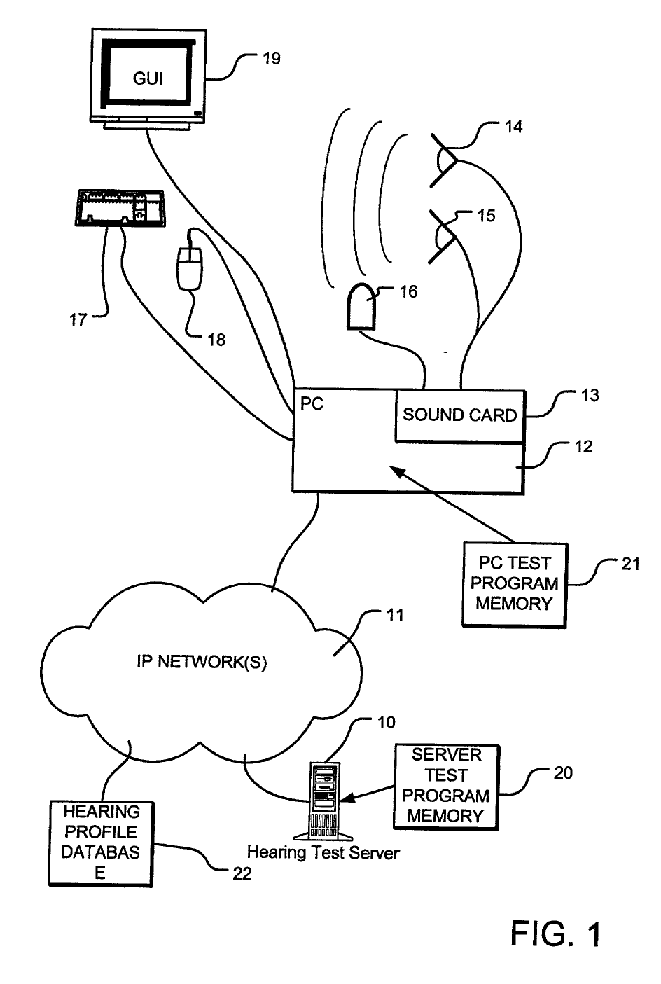

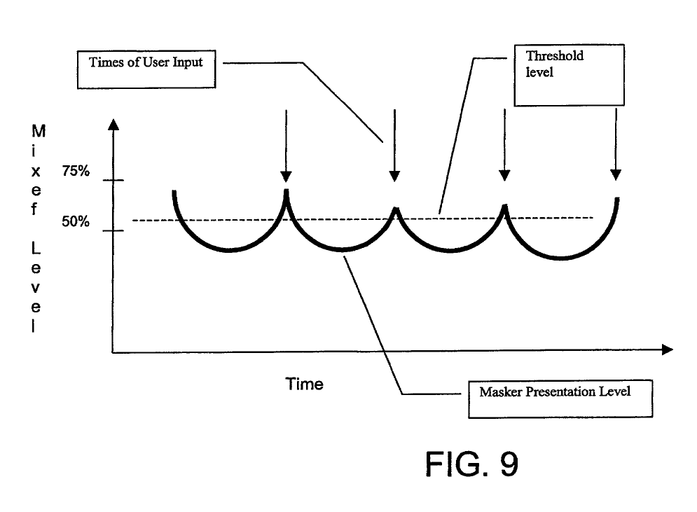

A detailed description of the various embodiments of the present invention is provided with reference to FIGS. 1-28A/28B.

FIG. 1 illustrates the Internet based system of the present invention implementing a hearing assessment test. System includes a remote calibration and hearing test server 10 coupled to a communication network 11, such as the Internet. The calibration and hearing test server 10 executes an interactive, calibration process and a converging hearing test protocol, such as the N-Alternative Forced Choice with a staircase convergence process described herein. A user end station 12, such as a personal computer, is also coupled to the communication network 11. The end station 12 includes a sound card 13 which provides data processing resources for producing audio output and receiving audio input under control of the logic in computer programs executed by the processor in the end station 12. In the figure, the sound card 13 is connected to stereo speakers 14 and 15, or to a headphone, and to a microphone 16. However, a wide variety of configurations exist in the end stations, which are not in the control of the calibration and hearing test server 10. The end station 12 also typically includes a display 19, a keyboard 17, and a mouse 18. During the tests, audio stimuli in the form of sound signals produced in the sound card 13 are generated using the stereo speakers 14 and 15 in this example. The sound signals may be sampled or computed sound. Environmental factors such as background noise, and the level of the output of the speakers 14 and 15 could be sensed using a microphone 16. The display 19 is used to display a graphical user interface which prompts a user to input data using the keyboard 17 or the mouse 18 in response to the audio stimuli of the test.

The calibration processes and hearing test are executed using a computer program that includes a first component stored on the server test program memory 20 which is connected to the server 10, and a second component which is stored in the PC test program memory 21 which is connected to the end station 12. Upon completion of a test, a calibration and a hearing profile are produced for the user. In a preferred system, this hearing profile is stored in a hearing profile database 22 which is accessible using Internet 11. In another embodiment, the hearing profile database 22 is coupled directly to the server 10. Alternatively, the hearing profile might be stored only on users end station and not made available to the communication network.

In this example, the end station 12 consists of a personal computer with standard s sound card components. In various embodiments, the end station consists of a mobile phone, hand-held computing platform like a personal digital assistant, or other consumer electronic device, like home stereo or television equipment having the capability to communicate with a remote test server.

In one implementation, the calibration and hearing test server 10 maintains a web site. To initiate a hearing test, a user at the end station 12 accesses the web site and downloads a component (e.g. a web page with or without active code, a .wav file that encodes an audio stimulus, or other software component) of the hearing test computer program from the server 10 for execution at the end station 12. The user initiates the test without intervention by a third party, and uses the resources available via the Internet and the resources at the end station to conduct a hearing test.

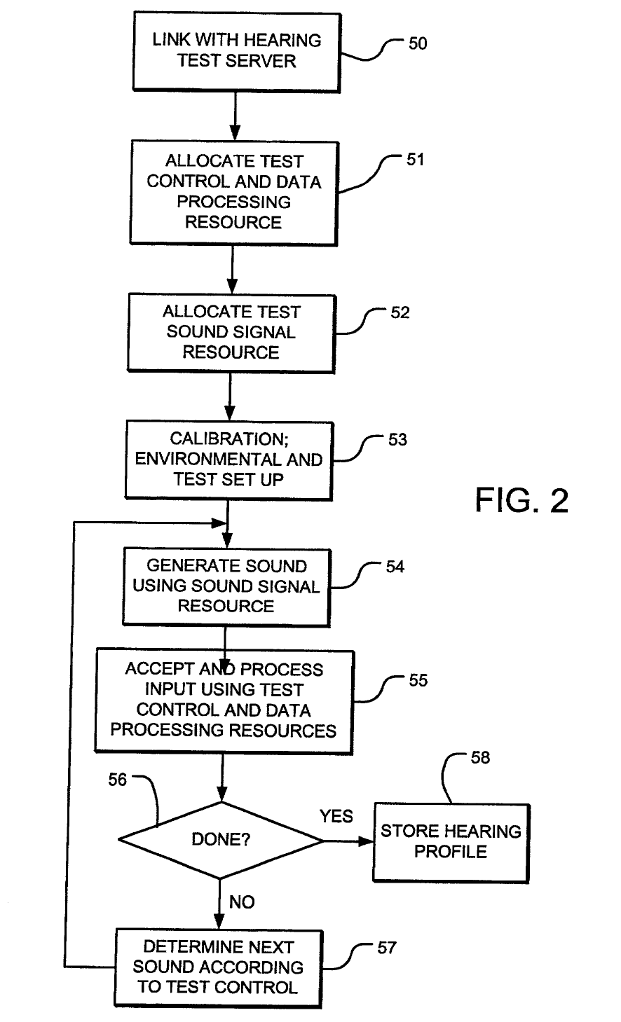

FIG. 2 illustrates the basic flowchart for the process of performing an Internet based calibration and hearing assessment test. In a first step 50, a user establishes a link between the end station and a calibration and hearing test server via a communication network such as the Internet. In one example, the link comprises a connection according to the transmission control protocol executing over the Internet protocol TCP/IP. The link may also involve protocols like the hypertext throughput protocol HTTP, and other Internet protocols. The link may be a wireless link via a cellular telephone network or a pager network.

In a next step 51, test control resources and data processing resources that will be utilized during the tests are allocated. The allocation of these resources can take a variety of configurations, including maintaining all of the resources at the server, and providing an Internet based interface accessible using a browser or email client at the end station, maintaining the test control resources at the Internet server, and data processing resources at the end station, or other combinations as suits the particular implementation of the program to control the test and to process the data generated during the test.

In step 52, test sound signal resources are allocated. The sound signal resources may include sound samples, programs for generating sounds, or other common sound synthesis tools. The sound signal resources are adapted to the particular type of hearing test to be executed. In one embodiment, the sound signal resources are downloaded to the end station from the server. In another embodiment, the sound signal resources are available in the personal computer sound card without requiring download from the server, such as by providing recorded audio files with drivers for sound cards that are loaded on a user's end station. In another embodiment, sound signal resources are distributed between the end station and a server during execution of the test.

Next, a calibration process according to the present invention is executed, involving the use of an item likely to be available to the test subject for producing a calibration sound, as described in more detail below (block 53). Optional other calibration programs are executed to evaluate the test environment (the audio environment in which the end station is situated), and the test set up (the audio characteristics of the equipment at the end station) to provide a baseline signal level for the device.

Upon completion of the allocation of data processing resources and calibration, test control resources and sound signal resources necessary for supporting the test, the test is initiated. The first step in the test is present an interactive interface to the test subject, including visual effects in N intervals, and to generate a sound using the sound signal resource in at least one of the intervals (block 54). Next, the process accepts and processes input using the test control and test data processing resources, by which the test subject signals a response selecting one of the N intervals as the one meeting the test criteria, such as whether the test subject heard the sound in this interval. (block 55). Next the routine determines whether the test has been completed, applying statistical analysis of the responses which indicate convergence on a result (block 56). If the test is not completed, then the algorithm determines a next sound according to a convergent test protocol (such as a “staircase protocol”) using the test control resources, in response to the input from the user and the state of the test (block 57). Then, the process loops back to step 54 to generate the next sound. If at block 56, it is determined that the test is completed, then the hearing profile is stored (block 58).

There are numerous options for prompting feedback and receiving signals from a test subjects. Options include accepting input in the form of the keystroke, a mouse click, use of a selection button or a timeout interval as volume is adjusted by the test control resources, or by the test subject action of increasing the volume, until some criterion is reached. A second option for accepting input includes causing the user to complete in action prompted by the graphical user interface, including graphical constructs which indicate respective test intervals, when test generated sounds meet some criterion during the respective intervals. For example, the test sound may be a sound varying in loudness. The test subject enters a mouse click when the sound disappears, or if it disappears in one of the N intervals. In another example, the test sound is played in one interval and not in a next. The test subject indicates the interval in which the sound is heard.

The test control resources can be distributed between the server and the end station using an Internet link, or using an executable file downloaded from the server and run locally on the test subject's equipment, or any partitioning of control in between. By controlling the test flow, the program can provide expertise for measuring and evaluating the level of background noise, testing for variability in the data, and in general control the flow and pace of the test process according to a test protocol. Controlling the test flow has specific advantages toward maintaining test subject interest as the user can be prompted to provide appropriate feedback and responses.

In other embodiments, data collected during the test can be returned to the web site server as raw data, as completely analyzed result data such as a hearing profile, or as any combination of raw and processed data in between. In one embodiment, data is not returned to the web site server in all, but rather completely processed locally on the end station using resources downloaded partially or completely from the test server.

The sound signals used in the testing process are implement in several alternative forms. The type of test signals used can have significant influence on the results of the test through a number of psychoacoustic effects. A large number of possible test signals are applicable to any of the implementations. Examples of the types of test tones claimed are:

Pure tones of long duration and constant intensity in each test step utilizing a number of different test steps at different frequencies.

Pulses of pure tones and constant intensity in each step utilizing a number of different test steps at different frequencies.

Combination of tones of long duration and varying intensity in each test step utilizing a number of different test steps at different frequencies.

Pulses of combinations of tones and varying intensity in each step utilizing a number of different test steps at different frequencies.

Constant amplitude, swept frequency sound in each test step utilizing different test steps at different amplitudes.

Constant amplitude pulses of swept frequency sound in each test step utilizing different test steps at different amplitudes.

Bandpass filtered noise combined with test signals.

Speech sound with and without noise background with or without temporal compression or elongation.

Furthermore the method of test sound signal generation is not limited, and can include sampling using standard formats like MIDI, FM synthesis, wavetable synthesis or other sound generation techniques.

As mentioned before, a wide variety of hearing test protocols can be utilized for producing a hearing assessment. The particular test chosen depends on a variety of factors, including the use to which the hearing profile will be put, the type of equipment used at the end station, and any information about the physiology of the test subject which may affect the choice of bearing test. Example test types include:

1) Hearing Threshold Level

The hearing threshold level test is related to identifying the sound level when the test subject can just begin to hear the test signal. This test type may be associated with determining the actual sound pressure level SPL of thresholds across the frequency range or the test method be simply to establish the relative level of thresholds as a function of frequency.

2) Masking Threshold Level

The masking threshold level identifies the test signal sound level when the test signal can be heard out of a masking signal. The masking threshold test protocol can be completed at a number of different baseline amplitudes to give an indication of recruitment. This method may have some advantages when there is some background noise at frequencies other than the test frequency.

3) Loudness Matching

In a loudness matching method, the generated sound consists of two different frequencies. One frequency is considered a baseline and is constant throughout a test. The other sound, the test sound, has a variable frequency during the test. A measurement consists of determining the loudness of the test sound that matches loudness of the baseline sound as a function of frequency. The resulting measurements are used to generate an equal loudness curve. The difference between the equal loudness curve obtained here and the equal loudness curve for normal hearing populations gives the hearing loss assessment. This test protocol can be completed at a number of different baseline amplitudes to give an indication of recruitment.

4) Loudness Growth in Octave Bands (LGOB)

Loudness Growth in Octave Bands is a subjective loudness evaluation procedure in which the test subject is prescribed set of common adjectives (e.g. very quiet, quiet, comfortable, loud, very loud and uncomfortably loud) to “measure” the loudness of test signals. The difference between the perceived loudness reported by the subject and a population of normally hearing individuals gives a measure of recruitment.

5) Speech Reception Threshold and Speech Discrimination in Noise or Quiet

These test methods are based on the fact that different speech sounds have different frequency spectra and so the speech reception/discrimination capabilities of a subject are dependent on the subject hearing profile. Furthermore, noise can be used to test the breadth of the auditory filter. Tests with background noise are particularly interesting for internet administered test because the controlled noise level can be set to mask the environmental noise.

6) Temporal Masking

Temporal masking of speech signals or tones can be used to probe auditory capabilities since it is known that temporal masking is affected by sensineural hearing impairment.

The test methods outlined above could be implemented in either a monaural or a binaural configuration. In the monaural implementation, each ear is tested individually and the other ear is “plugged” or otherwise deprived of test signal input. In an implementation scheme in which the headphones are supplied, the supplied headphones may have only one speaker. Clearly, there are advantages and disadvantages associated with either test method implementation with respect to accuracy and test complexity.

The basic test methods outlined above can be implemented within a number of different test configurations. The different test configurations may have different peripheral equipment, test protocols and they may have different levels of accuracy.

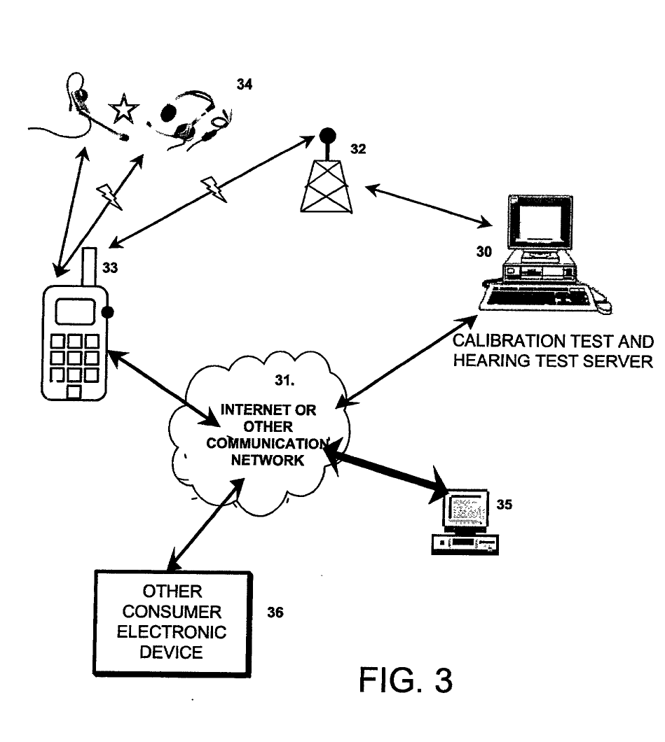

FIG. 3 provides a perspective of a variety of other types of remote devices which are suitable for use as end stations for calibration and hearing tests according to the present invention. A calibration and hearing test server 30, configured in a preferred embodiment for an N-alternative forced choice test, is coupled to the Internet 31 or other communication network, and via a wireless link to a cellular station or other up link station 32 which may support for example a cellular telephone network or a pager network. Mobile phones 33 with or without peripheral devices 34 like headsets and microphones, communicate via the up link station 32 with the server 30. A personal computer 35 may be coupled via the Internet 31 to the server 30, and act as an end station for the test. Other consumer electronic devices 36, such as stereo equipment or televisions, which are equipped for interactive communication via the Internet 31 or other types of communication networks, are also used as end stations at which test subjects perform the hearing tests of the present invention.

According to one embodiment of the present invention, an N-Alternative Forced Choice Procedure is executed using an interactive interface on the consumer electronic device. Forced choice procedures eliminate user bias by forcing the listener to choose between right and wrong alternatives. With this, each trial consists of several successive intervals of sound or sound presentations. These sound intervals are usually associated with a visual cue that is presented during the sound presentation and a visual representation representing each individual sound interval. The listener then selects one of the N intervals according to the criterion that they have been instructed. For example, they may have been instructed to select the interval that has a tone, where the other N−1 intervals had no sound. Or they may have been instructed to select the one interval that is different from the other N−1 intervals.

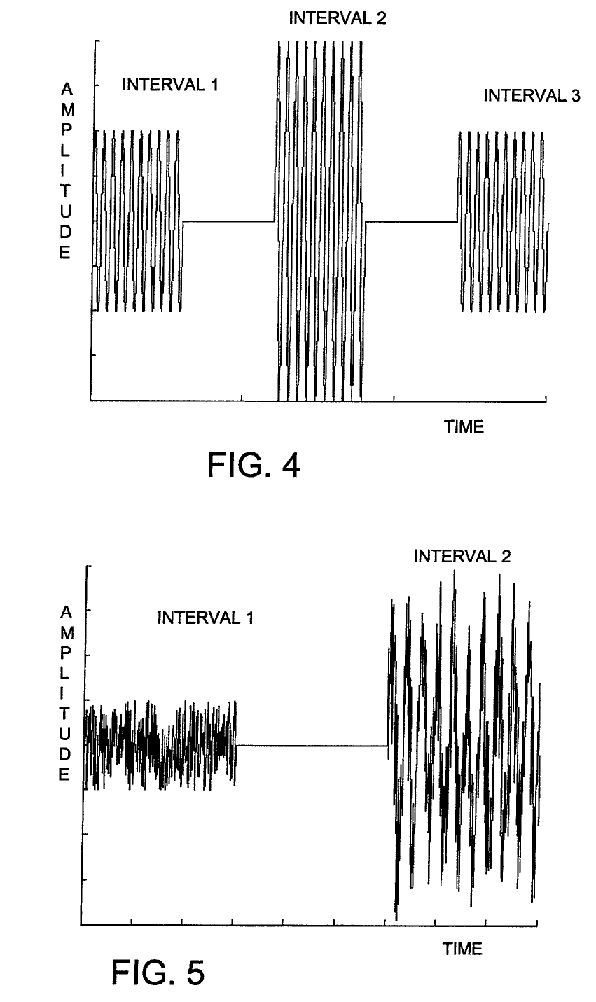

FIG. 4 is a plot of amplitude versus time, that shows tones produced for a 3-Interval Forced Choice where the listener is instructed to choose the interval that is different; here, the correct selection is Interval 2 which has a tone that is higher in level than the tones in Interval 1 or Interval 3. FIG. 5 is a plot of amplitude versus time, that shows tones produced for a 2-Alternative Forced Choice procedure where the listener has been instructed to select the interval which has a tone in the presence of noise; the correct answer is Interval 2, where Interval 1 has noise but no tone.

A convergent protocol for managing the test in one embodiment is an adaptive tracking procedure that meets accepted psychological standards. The adaptive tracking procedures described here are well known in the scientific auditory community but have not been used in web-based, or other remote hearing-loss measurement procedures. The first procedure, known as a staircase function, is an X-Down, Y-Up procedure where for every X incorrect responses, the task is made more difficult, and for every Y correct responses, the task is made easier. If the task is to detect a tone, X incorrect responses would result in an increase in the level of the tone for the next set of trials; Y correct responses would result in decreasing the level of the tone for the next responses. Both the correct and incorrect counts are reset to zero whenever the X or Y limit is reached. The method adaptively tracks to a specific percent-correct threshold, the value of which depends on the values of X and Y. For example, a 2-down, 1-up procedure adaptively finds the 70.7% correct point, while a 3-down, 1-up procedures finds the 79% correct point. This allows different thresholds to be estimated, depending on criterion such as number of trials wanted and performance level at which the user should hover around. The test continues either until a total number of trials has been reached or a total number of reversals has been reached. A reversal occurs for some tests when the adaptive procedure makes the test more difficult when the previous change had been to make the test easier, or when the test is made easier when the previous change was to make the test more difficult. For example, a reversal occurs when the adaptive procedure increases or decreases a sound level when the previous change had been in the opposite direction.

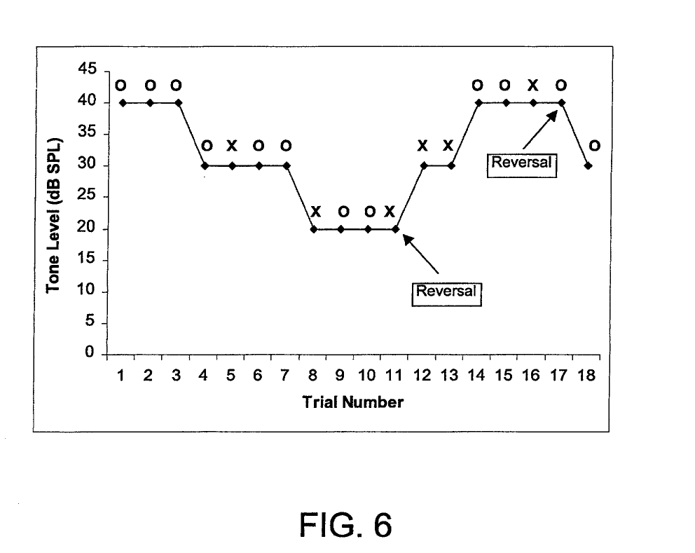

FIG. 6 is a plot of tone level versus trial number, that shows a run that used a 3-down, 2-up staircase procedure. Each symbol represents a trial where the listener had either a correct (O) or incorrect (X) decision. The abscissa indicates the trial number and the ordinate represents the level of the tone that is being adjusted according the listener. Table 1 details each trial of the run.

TABLE 1

| | | | | Change Level? | Current | Next | |
|---|---|---|---|---|---|---|---|
| Trial # | Response | #Correct | #Incorrect | Direction | Level | Level | Reversal? |
| 1 | Correct | 1 | 0 | No | 40 | | |
| 2 | Correct | 2 | 0 | No | 40 | | |
| 3 | Correct | 3 | 0 | Yes, Decrease | 40 | 30 | No |
| 4 | Correct | 1 | 0 | No | 30 | | |
| 5 | Incorrect | 1 | 1 | No | 30 | | |
| 6 | Correct 2 | 1 | No | 30 | | | |
| 7 | Correct | 3 | 1 | Yes, Decrease | 30 | 20 | No |
| 8 | Incorrect | 0 | 1 | No | 20 | | |
| 9 | Correct | 1 | 1 | No | 20 | | |
| 10 | Correct | 2 | 1 | No | 20 | | |
| 11 | Incorrect | 2 | 2 | Yes, Increase | 20 | 30 | Yes |
| 12 | Incorrect | 0 | 1 | No | 30 | | |
| 13 | Incorrect | 0 | 2 | Yes, Increase | 30 | 40 | No |
| 14 | Correct 1 | 0 | No | 40 | | | |
| 15 | Correct | 2 | 0 | No | 40 | | |
| 16 | Incorrect | 2 | 1 | No | 40 | | |
| 17 | Correct | 3 | 1 | Yes, Decrease | 40 | 30 | Yes |
| 18 | Correct | 1 | 0 | No | 30 | | |

An alternative to the up-down staircase tracking procedure is the maximum likeihood Procedure. See, Green, “Maximum likelihood procedures and the inattentive observer,” J. Acoust. Soc. Am. 97(6), June 1995, pp. 3749-3760. The maximum likelihood procedure assumes a form of the psychometric function (for example, percent correct as a function of the signal characteristic that is being adapted, such as level of a tone) and calculates the most likely psychometric function based on Bayesian statistics. There is the suggestion that this procedure is faster than an up-down procedure, but this is still being debated. This maximum likelihood procedure has also been applied to yes-know tasks in controlled environments.

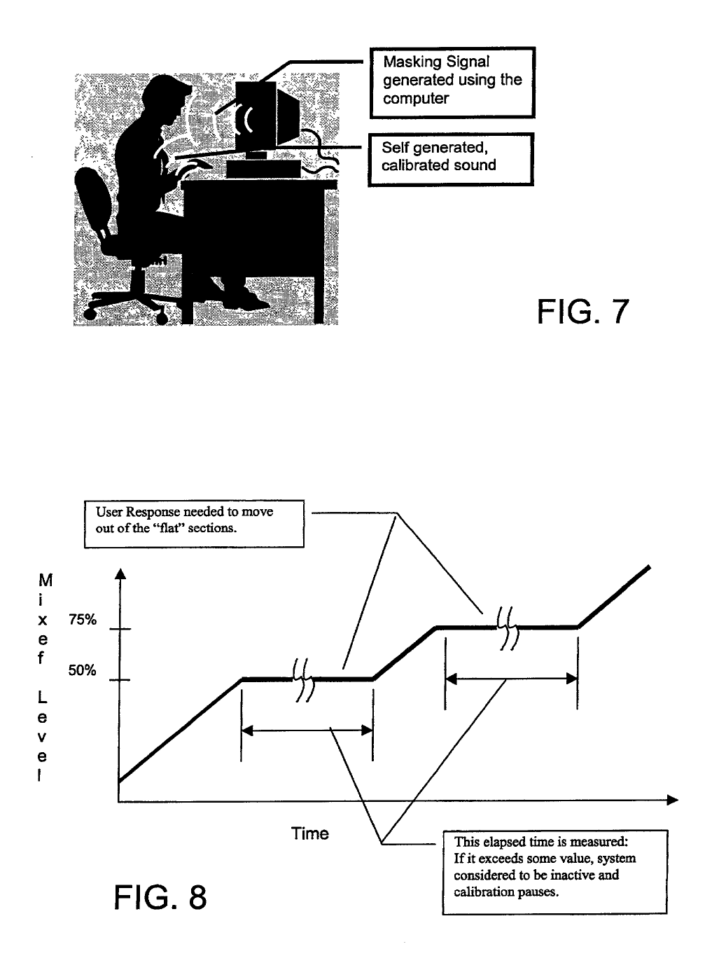

A calibration module “calibrates” a computer system sound card, or other audio resources at the consumer electronic device in a remote site, by generating a setting of programmable volume controls on the electronic device matching a calibration sound within a usable threshold. A usable threshold in one embodiment, allows for production of sounds for use in a hearing test that have actual sound pressure levels within a high probability between 5 dB and 10 dB of predicted level. FIG. 7 shows the basic calibration concept according to a preferred embodiment. A test subject 60 at a remote device 64 produces a self-calibrated sound 62, while the audio resources are used to produce a masking signal 63. The calibration module is based on determining the computer mixer levels needed for a white noise signal, or alternatively a masking sound designed to generally match the calibration sound, to just mask (masking threshold) a calibration sound generated at the remote site by the test subject. Since the level of the noise generated at the remote site will be known within a range, the sound card mixer levels and the encoded amplitude of the sound file associated with the masking threshold will be understood to define a sound pressure level within a range for white noise, or for a noise matching the calibration sound. Although a masking test is preferred, other calibration tests, such as loudness matching, may be used in some embodiments using the prompting and calibration sound generation techniques of the present invention.

In one embodiment of this procedure, the system automatically drives the mixer slider levels up until a user, through an input from the keyboard, indicates that the level of the masker has crossed through the masked threshold. Upon this input, the slider levels are automatically driven low, below the threshold level and subsequently back up toward a level slightly above the previous reversal. Again a user input indicating the threshold crossing, is used to halt the upward travel of the slider level. Multiple reversals are used to increase the accuracy of the estimate of the threshold.

The user will generate, using common items, a sound called the calibration sound. From study of the noises generated, the range of the mask noise levels needed to mask the sound will also be known. Examples of the specific noise generated, among many possible noises, include the following:

Striking the keyboard

Making circles on a piece of paper with a pencil

Rubbing two pieces of paper together.

Many different types of sounds are suitable for the calibration sounds. The following outlines the groups and gives some examples within the groups:

Impact:

Something tapping on a surface

Pencil on a book

Finger on a desk

Stapler stapling

Keyboard of computer

Computer mouse “click”

Dropping object:

Coin on a coin

Coin on a piece of wood

Pen/pencil dropping on a book

Jingling:

keys on a key chain

paper clips

Rubbing/Friction:

Hand on paper

Paper on paper

Pencil writing on paper

Coin on a phone book

Vibrational sounds:

String plucking

Rubber band vibrating

Ruler vibrating

Some sounds will be more easily repeated across people than other sounds. The more repeatable the sound the better. Some factors involved in making a sound more repeatable are:

Involving some standardized components in the sound making mechanism. Examples of standardized components would be coins, pencils and paper where the standardized features of importance are weight and composition, composition and surface and surface finish respectively.

Generated sound should be repeatable many times. This means that the act of making the sound should not destroy the sound making mechanism. Examples of sounds that destroy the mechanism are breaking sounds, paper crumpling and opening.

Because of the limited power output of computer systems, the sound should not be too loud. Further, since many people have hearing problems, the sound should be sufficiently loud and its dominant energy should be below ˜5000 hz.

The masker is, in one embodiment, a full band white noise (random numbers generated in the time domain). Alternatively, the masking sound is produced so that it has a spectral content resembling that of the calibration sound. Throughout the calibration module, the presentation of the masker will be adjusted through adjustments made, for a personal computer running the Microsoft Windows operating system, to the Wave Out (also called All Wave) slider and the Master Volume slider of the Windows Sound Card Mixer. These sliders will be adjusted simultaneously.

A masking sound which is a true, fullband white noise is generated through the generation of random numbers. The RMS power will be −5 dB bit. The relationship between dBpower and dBamplitude for white noise is:

dBpower=dBamplitude−4.75 dB

The masking sound can be either white noise or shaped noise. Using shaped noise can have the affect of reducing the power needed to mask any particular narrow band sound. In addition, spectrally shaping the masking noise to match the spectrum of the calibration sound reduces the dependency of the calibration level set during the test on hearing loss.

Since some of the users may be hearing impaired, it is important to use noises with similar bandwidths for both masker and the Calibration Tone. Hence, they can be both broadband or both narrow band but not both narrow and broadband.

It is advantageous to use a calibration sound and masker sound that are, when played separately individually identifiable. When the two sounds get to be too similar, then the masking task is as much a matter of sound localization as it is about masking.

In one alternative approach, the two noises are reversed: if the calibration sound is a broadband sound, then the “masker” can be an octave band noise. Now, the point of interest is the “release from masking” point: the point where the octave band noise can be “heard out” from the masking of the broader band calibration noise.

During the presentation of the masker, the presentation level of the masker will be modulated according to a predetermined algorithm that uses input from the user. The predetermined algorithm includes a ramp-up phase and a test phase. In the ramp-up phase the general region of slider position that corresponds to the masking threshold is determined and some protections are installed to ensure that the overall sound level can not get to maximum slider levels without direction from the user. In the test phase, the sliders are modulated from the reversal point, down below the threshold and then back through the threshold to 50% to 90%, for example, of the previous reversal value(s).

An effort to avoid allowing the mixer settings of a potentially loud system to be driven to their maximum values without user input, the ramp-up phase will include a number of regions where the level is not increased without further input from the user. FIG. 8 shows a schematic of the ramp-up phase. The various slopes and durations depend on particular configurations and design choices.

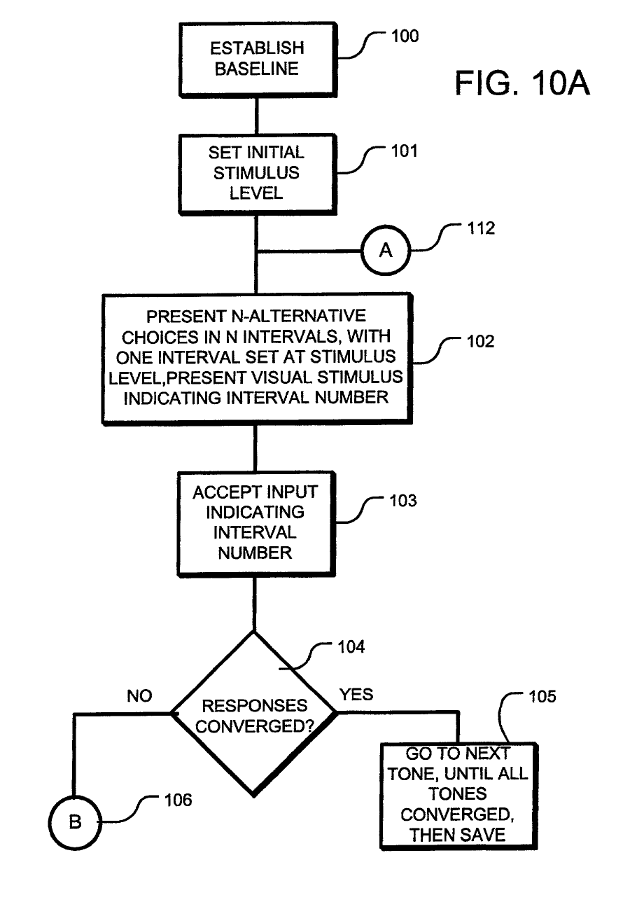

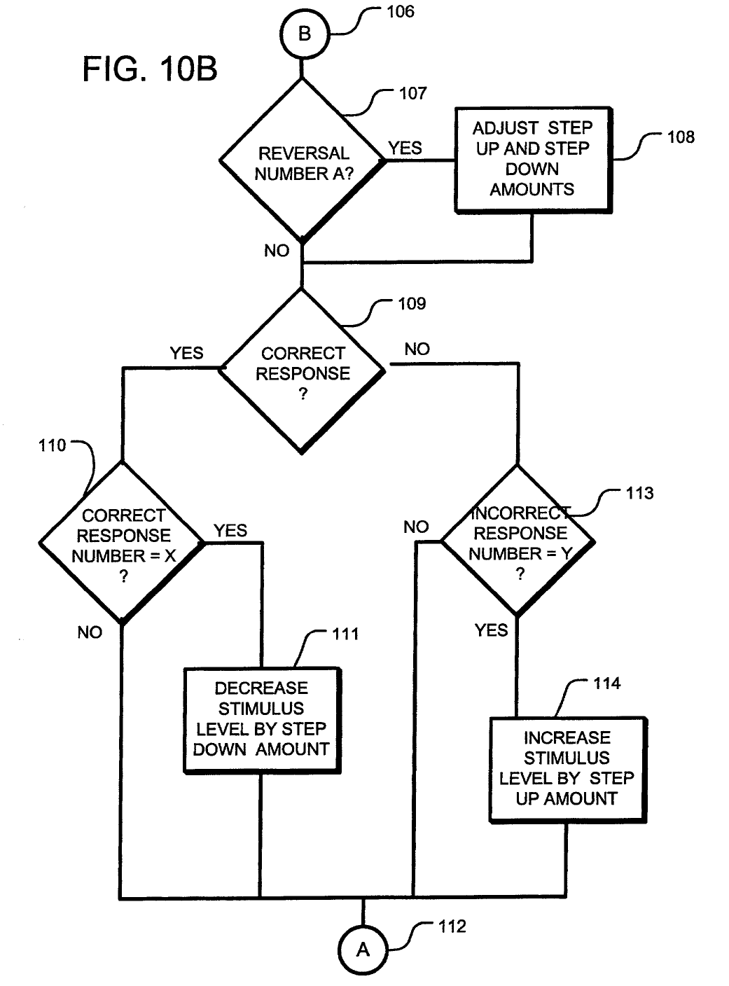

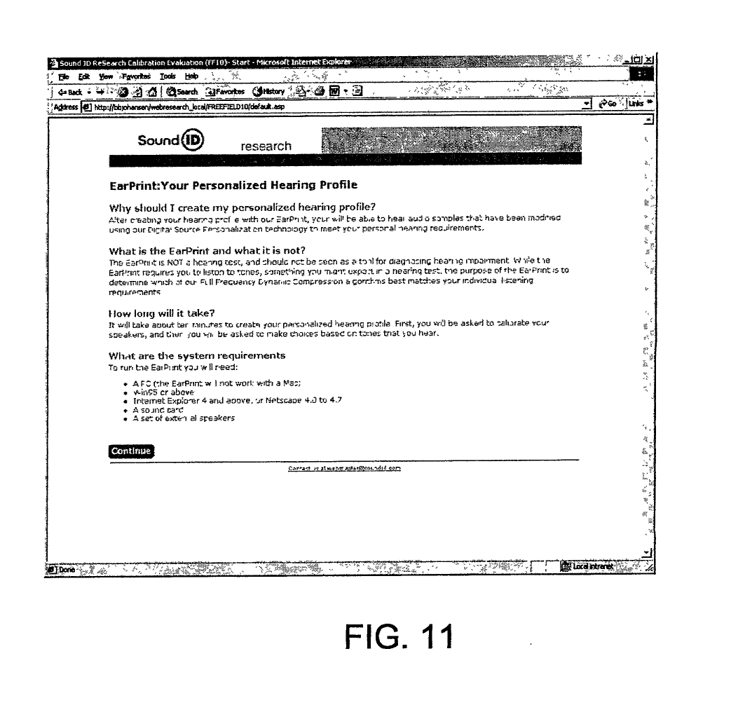

During the test phase, the mixer slider level automatically cycles from above the threshold to below the threshold to back above the threshold. A new cycle is initiated by the user input that indicates that the threshold has been crossed. FIG. 10 is a plot of a mixer level versus time, with a trace 70 of the masker sound level as it traverses a threshold level 71, and reverses after user inputs at times 72, 73, 74 and 75. Specific signal levels and slopes of the traces are determined based upon empirical analysis.

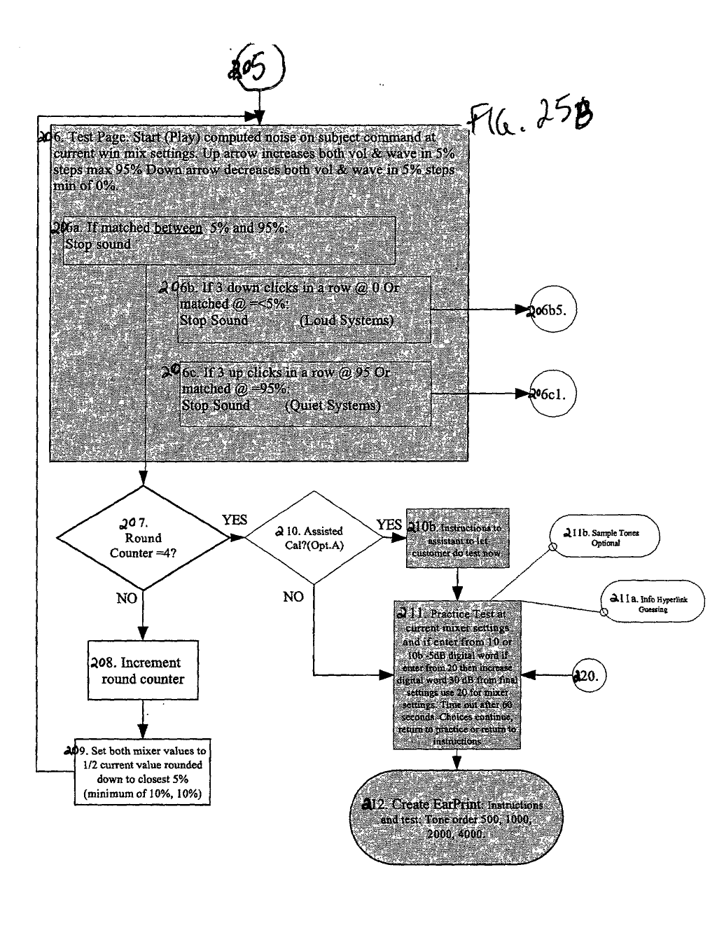

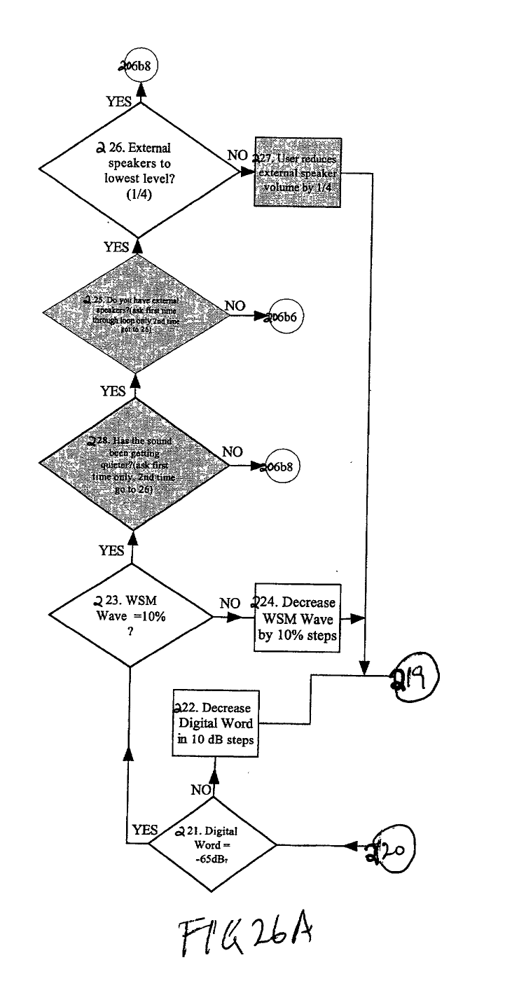

In an alternative embodiment, the user is instructed to manually adjust the mixer volume controls during the calibration test. A detailed example of the manual flow is provided below with reference to FIGS. 25A and 25B.

The adjustment of the masker to find the masking threshold can be done in a number of ways:

Computer operator can simultaneously adjust the volume, through any variety of volume controls while making the calibration sound.

A computer program can drive the sound level along a pre-prescribed algorithm that adjusts itself based on inputs from the user.

The Masker can be held at a constant level while the computer operator compares the calibration sound. Then the operator, through the use of a keyboard or mouse entry requests either a louder or softer masker level. This process is repeated until the operator is satisfied that the masking threshold has been found.

Embodiments of the present invention apply the principle of auditory masking as a basis for setting the output sound level of a remote system. Masking involves a determination of when the excitation pattern in the cochlea of the subject caused by the calibration sound is drowned out, or no longer sensed, because of the excitation pattern of the masking sound. Masking tests are superior to loudness based tests, more objective because the determination of whether the calibration sound can be sensed at all is more objective, and thus more repeatable, than a loudness comparison in which the subject is asked to state when two sounds have the same loudness. In the calibration method based on masking, the subject finds the masking level of a calibration sound, which is self-generated in some embodiments as described above, for a computer generated masking sound. In one embodiment, the calibration sound is generated by rubbing two sheets of paper together on a flat surface. This sound has good repeatability properties across individuals and locations. A masking sound spectrally shaped to match the calibration sound is preferred for the following reasons:

Reduced power at masker level. Some systems that previously where unable to output sufficient power to mask the calibration sound should now be capable of masking the calibration sound.

Reduce potential interactions between hearing loss and calibration sound spectral shape on the level set of calibration.

Another feature of the masking noise's spectral content is the affect speaker frequency response has on the actual dBSPL/Hz distribution expressed. Measurements indicate that the frequency response of typical speakers falls off somewhere above 4 khz. This speaker response can have implications on the variability of the resulting calibration since the masker level will need to be raised artificially high to mask the considerable high frequency energy present in the current calibration sound. In this situation, the response of the speakers will, in effect be setting the level of the calibration. As a result, it is advantageous to increase the spectral energy in the high frequency region so that the high frequency content of the calibration sound is masked, even in the face of speaker roll-off, long before the calibration sound components below about 4 khz.

The masking noise is a noise signal used to “drown out” the calibration sound. The masking noise is generated by the computer. The factors discussed above are included in defining the spectral shape of the masking signal, so that it matches the calibration sound to a degree sufficient for a reasonably accurate masking level test.

One embodiment of the spectral shape will be defined in terms of dB/Hz. Furthermore the spectral shape is specified in terms of normalized values since the overall level will be set by the subject. The spectral shape may be defined at a few frequency values. Linear interpolation of the dB/Hz values between the given values will be used to determine intermediary and limits. Smoothing of the resulting “shape” is not required. Values at or near DC are of little consequence since the output of typical computer sound systems at very low frequencies is attenuated. One example spectral shape is provided in the Table 2 below.

TABLE 2
Spectral Shape of Shaped Masker

| Frequency | | | | | | | | | | | | |
|---|---|---|---|---|---|---|---|---|---|---|---|---|
| normalized | DC | 125 | 250 | 500 | 1K | 2K | 4K | 6.3K | 8K | 10K | 13K | 20K |
| dB/Hz | <−80 dB | −18 | −18 | −18 | −12 | −6 | 0 | 0 | 0 | 0 | 10 | 10 |
| normalized | | | | | | | | | | | | |
| dB/Hz-MAX | −13 | −13 | −13 | −7 | −4.5 | 0 | 2.5 | 2.5 | 2.5 | 15 | 15 | |
| normalized | | | | | | | | | | | | |
| dB/Hz-MIN | −23 −23 | −23 | −17 | −8.5 | 0 | −2.5 | −2.5 | −2.5 | 5 | 5 | | |

The phase of the signal may be essentially random across the + or − pi range.

As a result of the calibration process, the value of digital signals sent to the computer to produce a sound level near that of the calibration sound is determined. This value is expressed for example as dB down from a digital maximum level. Thus if the calibration sound is known to be about 68 dB, and the value determined by the masking process is about −45 dB, then a sound pressure level that is close to 40 dB will be produced by a digital value corresponding to −73 dB from digital maximum.

FIGS. 10A and 10B together show a simplified flow chart of one version of the interactive, converging test protocol of the present invention. The test protocol according to one embodiment of the present invention finds a hearing threshold level for a set of tones, such as 500 Hz, 1 kHz, 2 kHz and 4 kHz. It begins with a process to establish a baseline signal level for the remote device (block 100), using a calibration procedure, such as that described above, or other calibration procedures which may involve the use of specialized hardware or other techniques for direct measurement of sound pressure levels at remote device. Various calibration procedures are described in the above referenced related patent application Ser. No. 09/830,480, INTERNET BASED HEARING ASSESSMENT METHODS, invented by Menzel et al., filed Apr. 26, 2001, which is incorporated by reference as if fully set forth herein.

After establishing a baseline, the test resources set an initial stimulus level for a particular tone in the set of tones to be used in the test (block 101). The initial stimulus level may be for example about 30 dB above a typical hearing threshold for a normal hearing test subject. Next, N-alternative choices are presented in N time intervals, with one interval set according to the selected stimulus level, while at the same time presenting visual stimulus indicating an interval number to the test subject (block 102). The visual stimulus may be provided using a variety of techniques, such as Internet web page “button” constructs presented on a display at the remote device, or even simple lights on the remote device, such as LEDs on a mobile phone. The intervals last in one embodiment between 300 and 700 milliseconds, for example about 500 milliseconds. The time between the intervals is preferably less than a than a second, and more preferably about 300 to 700 milliseconds, such as for example, 500 milliseconds. According to the protocol, input from the test subject is accepted indicating the interval number during which the selected stimulus is perceived by the test subject (block 103). The process determines next whether the responses have reached a stopping criterion indicating convergence on a result, such as by determining a percent correct parameter (block 104). If the responses have converged, then the algorithm branches to block 105, where the it proceeds to the next tone until all the tones in the hearing test have converged, and the test results are saved. If at block 104, it is determined that the responses have not converged, the process proceeds through B (block 106) to the process of FIG. 10B. Next, the number of reversals is determined (block 107). If the reversal number matches a number A, then the step up and step down amounts are adjusted (block 108). If the reversal number is more or less than A, or after block 108, the process determines whether the test subject provided correct response (block 109). If the response was correct, then the process determines whether the number of correct responses matches X (block 110) if the number of correct responses matches X, then the stimulus level is decreased by a step down amount (e.g. down by 10 dB initially and 5 dB after the number A reversals have been encountered) and the correct response number is reset (block 111). If the correct response number is less than X, or after block 111, then the process loops through A (block 112) back to the process at block 102 of FIG. 10A. If at block 109, it is determined that the test subject did not provide a correct response, the algorithm determines whether the incorrect response number matches Y (block 113). If the incorrect response number matches Y, then the process increases the stimulus level by a step up amount (e.g., up by 10 dB initially and 5 dB after A reversals have been encountered), and the incorrect response number is reset (block 114). If the incorrect response number is more than or less than Y at block 113, or after block 114, the process loops through A (block 112) back to the process at block 102 of FIG. 10A.

The parameters A, X and Y in the process of FIGS. 10A and 10B can be selected as suits needs a particular testing environment, and of a particular hearing characteristic being tested. For a basic hearing profile, X equals a number in the range of 2 to 6, and Y equals the number in the range of 1 to 4. For example, the test where X equals 3, and Y equals 1 is useful, providing a “three down, one up” convergence process. The parameter A falls preferably in a range of 2 to 5 for a basic hearing profile.

In a preferred embodiment, the parameter X equals 1, and the parameter Y equals 1, until the first reversal. (One down, one up). Thereafter the parameter X is changed to 3, and the parameter Y remains 1. (Three down, one up). It is found that the initial one down, one up stage speeds the convergence process.

Also, the adjustment of the step up and step down amounts may be allowed to occur only once in a given test procedure, or may be allowed to occur many times as suits in the needs of a particular process.

As mentioned above, an alternative adaptive, converging process for adaptively selecting the stimulus levels, and converging on a result is the maximum likelihood test, in which a statistical process is applied to predict a next stimulus level based on a likely threshold determined from a set of responses gathered during the test. A single false or erroneous response does not cause the program to presume convergence for maximum likelihood algorithm.

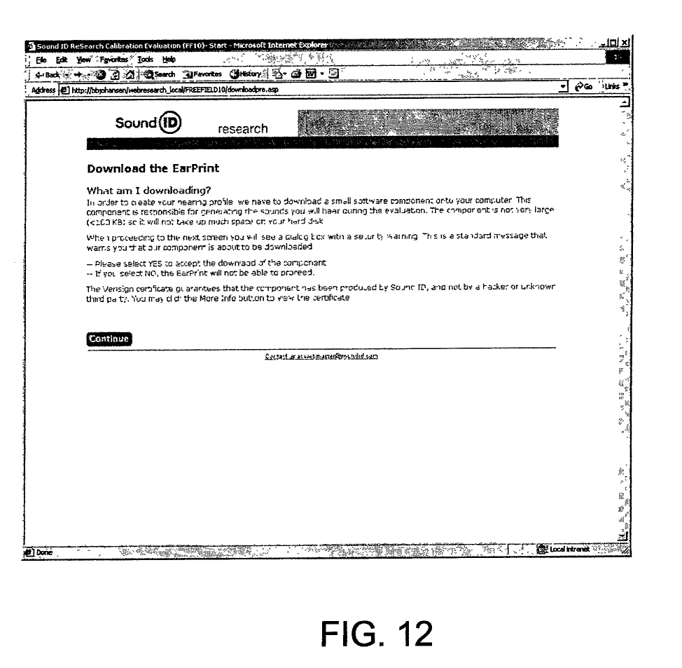

FIGS. 11 through 24 are images of web pages generated by a routine that causes presentation of a calibration test and an N-alternative forced choice hearing test providing interaction with a convergent procedure according to one embodiment of the invention. The web pages are rendered by a standard Internet browser, such as Internet Explorer provided by Microsoft Corp., in an interaction with the test server. An opening screen for this example is shown in FIG. 11. The opening screen of FIG. 11 introduces the concept of the hearing profile and explains system requirements to a test subject. If the test subject selects the “continue” button on the web page of FIG. 11, the page of FIG. 12 is presented, which prompts the test subject to allow downloading of a component of the hearing test program from the server for use in execution of the test. In this embodiment, the component downloaded comprises a routine, implemented for example as a DirectX file, for generating the audio stimulus for the test and calibration processes, for managing the interaction during the test and calibration processes, and for adaptively selecting the stimulus levels according to a staircase function as described above. The server continues to execute a component that maintains communication with the remote site, and reacts to messages from the remote site, such as receiving the results of the testing, and interacting with the test subject before and after the test.

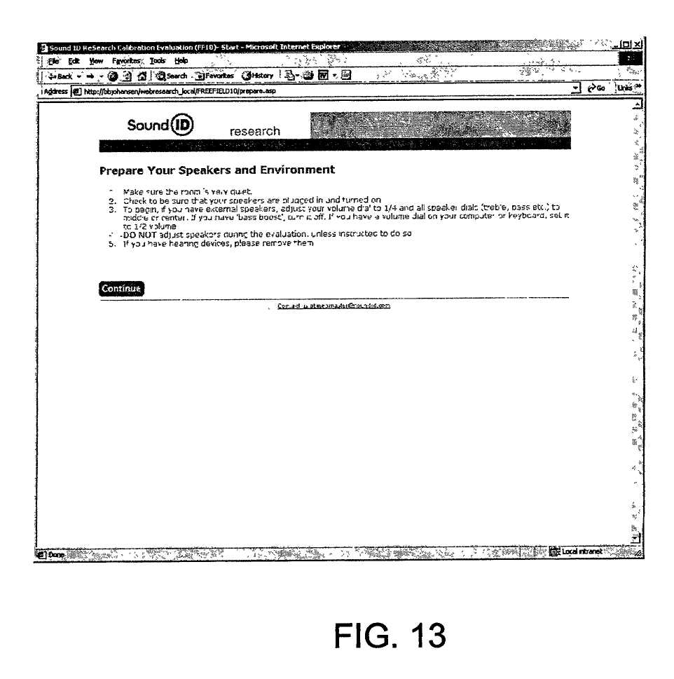

If the test subject selects the “continue” button on the web page of FIG. 12, then the component is downloaded, and the web page shown in FIG. 13 is presented. The web page of FIG. 13 prompts the user to prepare the speakers and environment for the test. This includes instructing the test subject to make adjustments of the audio parameters on the device, such as a personal computer, to be used during the test.

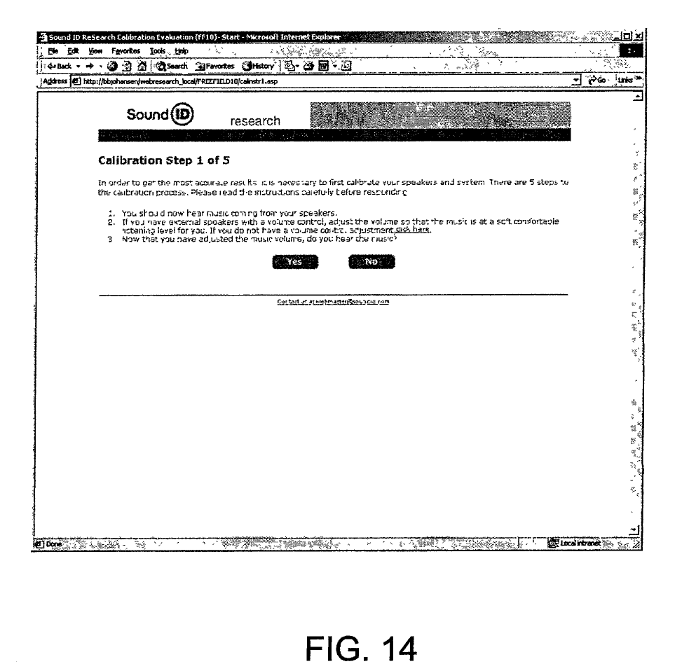

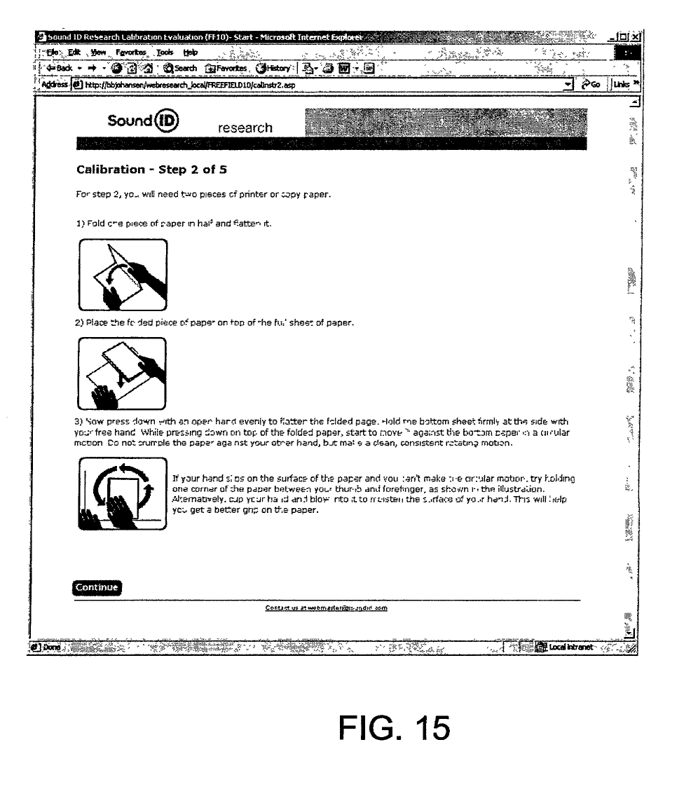

If the test subject selects the “continue” button on the web page of FIG. 13, message is shown to the user that a software component is being downloaded to support the calibration step. The component downloaded at this stage is a compressed audio file storing music. When the music file is downloaded, the web page shown in FIG. 14 is presented. The web page shown in FIG. 14 explains the first step in a calibration process. According to the first step, during presentation of the web page, the music file is played in the speakers. Users instructed to adjust the volume so the music is at a soft, comfortable listening level. If the user successfully performs this step, and selects the “yes” button in FIG. 14, then the web page shown in FIG. 15 is presented.

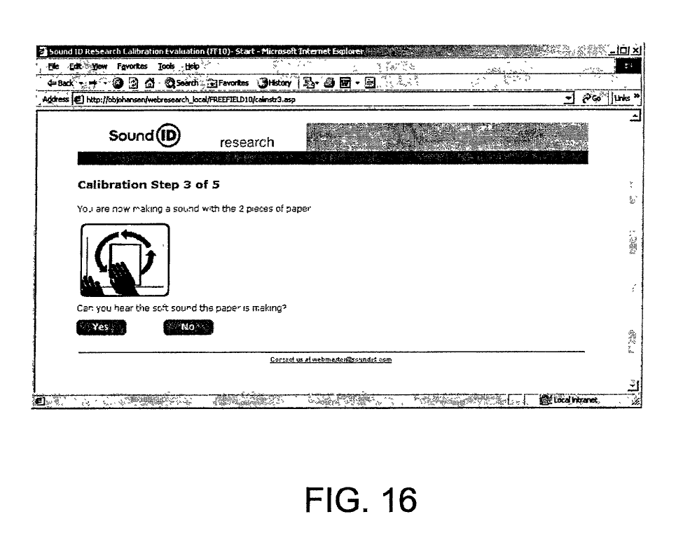

The web page shown in FIG. 15 explains the second step in the calibration process. During the second step, the test subject prepares a calibration sound source using ordinary items. In this example, the web page explains how to prepare to pieces of printer or copy paper so that the process of rubbing the paper together can be executed to generate a calibration sound. If the user presses the “continue” button on FIG. 15, then the web page shown in FIG. 16 is presented.

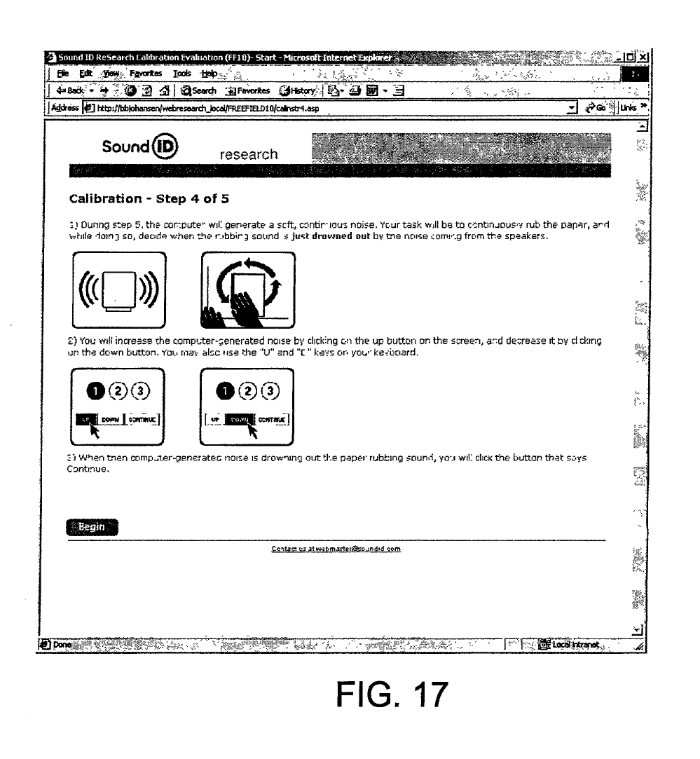

The web page shown in FIG. 16 prompts the test subject to verify that a calibration sound is being made using the items described in FIG. 15. If the test subject selects the “yes” button in the web page of FIG. 16, then the web page of FIG. 17 is presented.

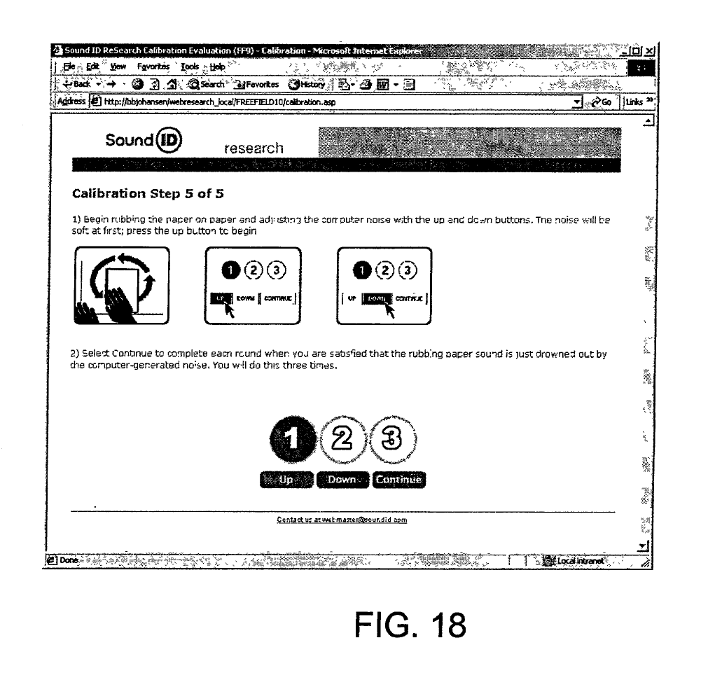

The web page of FIG. 17 illustrates and explains the process to be used in order to set a baseline level for the personal computer using the calibration process. Basically, the computer generates a soft, continuous noise. The test subject continuously rubs the paper together, and decides when the calibration sound is just drowned out by the noise coming from speakers. The test subject increases the computer-generated noise by clicking on a button in the screen presented during this process, or by using other input devices. Finally, the web page in FIG. 17 explains that when the computer-generated noise is drowning out the paper rubbing sound, then the test subject signals completion of the test by clicking the “continue” button to be presented during the test. If the user selects the “begin” button shown in FIG. 17, then the web page shown in FIG. 18 is presented.

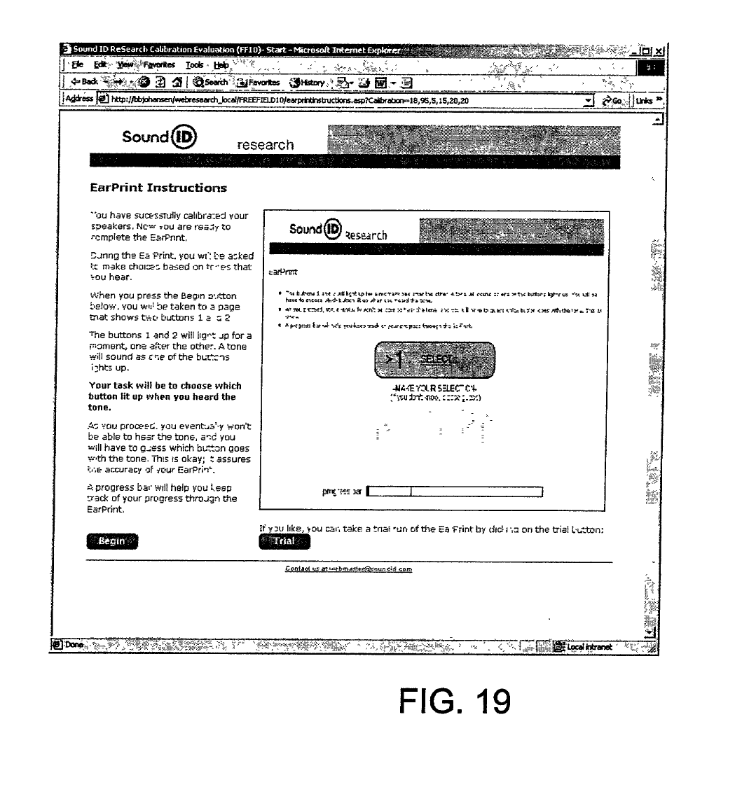

The web page shown in FIG. 18 is the last step in the calibration process, during which the test subject determines the level at which the computer-generated noise drowns out the paper rubbing sound. Thus, the web page shown in FIG. 18 prompts the user to begin rubbing on the paper and adjusting the computer-generated noise using the “up” button, and “down” button, until the masking level is reached. When the masking level is reached, then the user is instructed to select the “continue” button. The screen includes three indicators, which comprise the numerals 1, 2 and 3 within respective circles. When the test is completed a first time, the first indicator is highlighted. When the test is completed a second time, the second indicator is highlighted. When the test is completed the third and final time, the third indicator is highlighted. When the test subject selects the “continue” button after the third level setting process in the web page of FIG. 18, then the web page of FIG. 19 is presented.

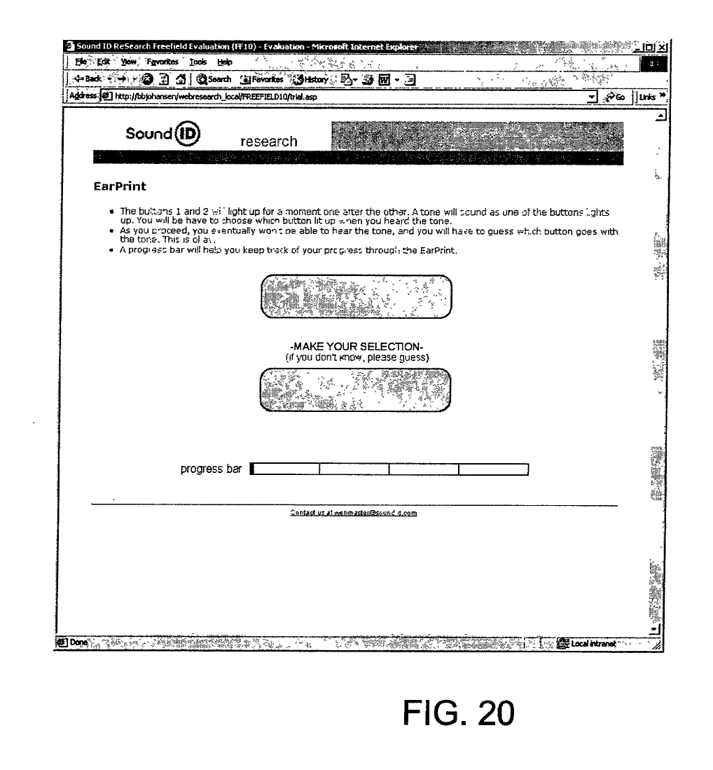

The web page of FIG. 19 represents the start of the N-alternative forced choice test, and explains the testing procedure. Thus, the web page of FIG. 19 explains that the test subject will be asked to make choices based on tones that he or she hears. In the example shown in FIG. 19, the test subject is offered the opportunity to run a trial by selecting the “trial” button in the web page of FIG. 19. If the user selects the “begin” button in the web page of FIG. 19, then the web page of FIG. 20 is presented. The buttons “1” and “2” in FIG. 20 will light up, or otherwise be highlighted, for a moment, one after the other. A tone will sound as one of the buttons lights up. The task of the test subject is to choose which button lit up when the tone was perceived. As the test subject proceeds, the test subject eventually will not be able to hear the tone and will have to guess which button goes with the tone. A progress bar keeps the test subject informed about progress of the testing. The buttons “1” and “2” are graphic constructs aligned in an up and down relationship, rather than a left and right relationship in this embodiment of the invention. It is found that the up and down relationship is preferred in environments in which test subjects may be mistakenly correlate the left button with a left speaker and the right button with a right speaker in a stereo configuration.

Using this interface, where the visual indicators of the test intervals comprise highlighting of the buttons “1” and “2,” the user is prompted through the testing procedure. The testing-procedure follows a process such as described above with respective FIGS. 10A-10B.

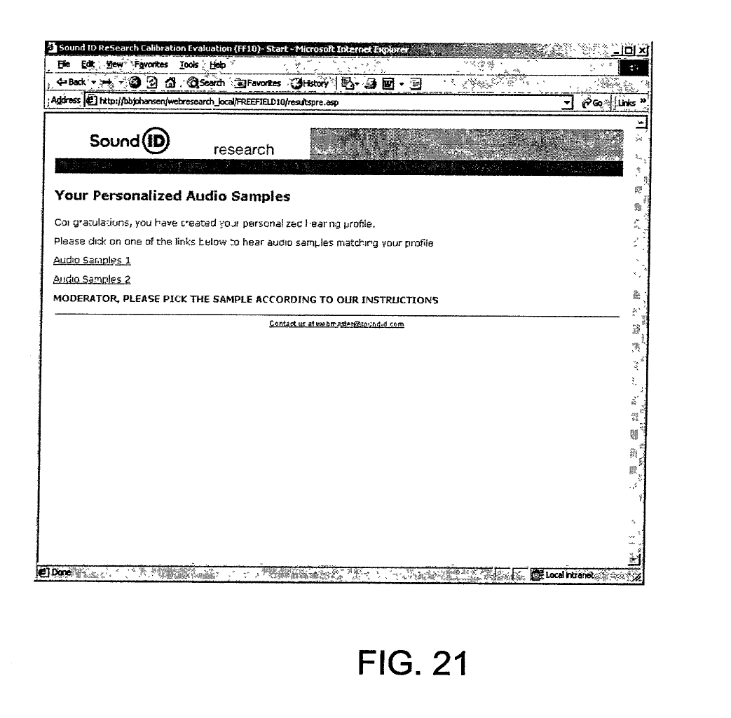

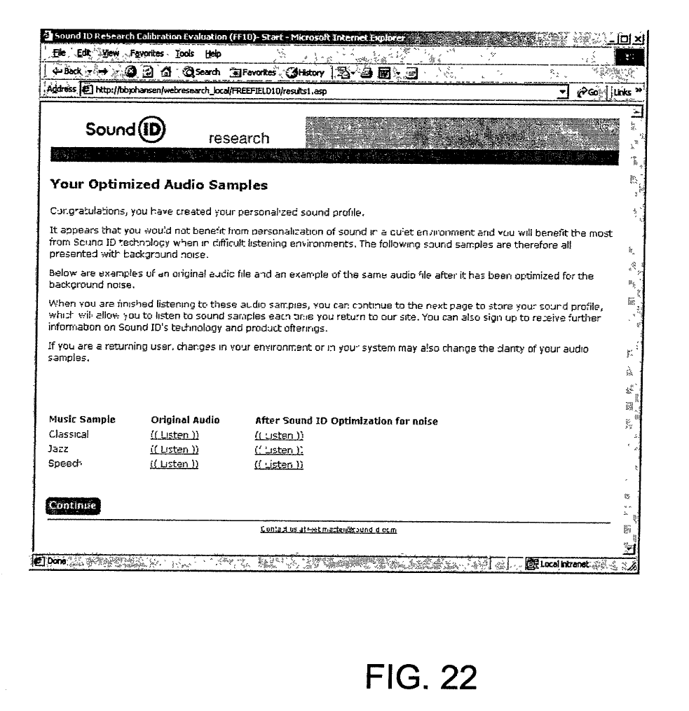

When the test is completed, either the web page shown in FIG. 21 or the web page shown in FIG. 22 is presented. The web page of FIG. 21 is presented if the hearing profile produced by the test suggests that the test subject could benefit from personalized audio generated by applying hearing profile. In the web page of FIG. 21, the test subject is prompted to playback audio samples which have been adapted according to the hearing profile created using the test. The web page of FIG. 22 is presented if the hearing profile of the test subject is within a normal range, suggesting that the hearing profile can be applied for personalized audio products in a noisy environment, but may not be necessary in a quiet environment. The user is prompted to select samples of audio products which simulate a noisy environment in a original format and in a optimized format.

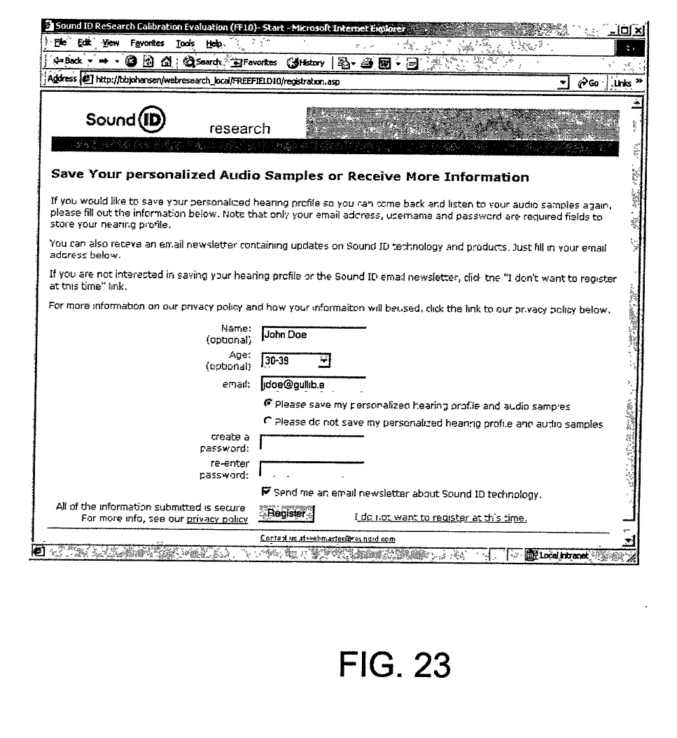

If the user selects the “continue” button in the web page of FIG. 22, then the web page of FIG. 23 is presented. The web page of FIG. 23 allows the user to register with the web site, store the hearing profile, and otherwise participate in activity supported for registered users of the web site.

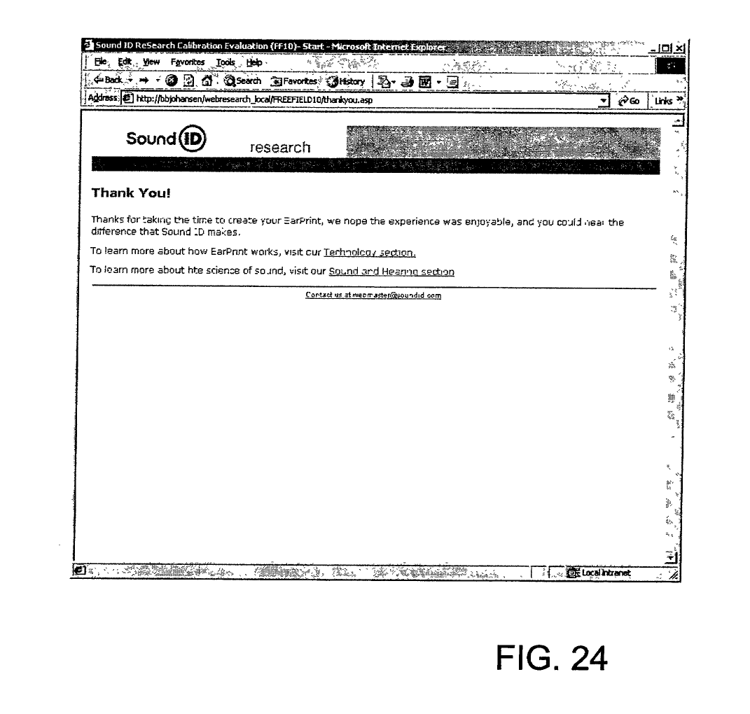

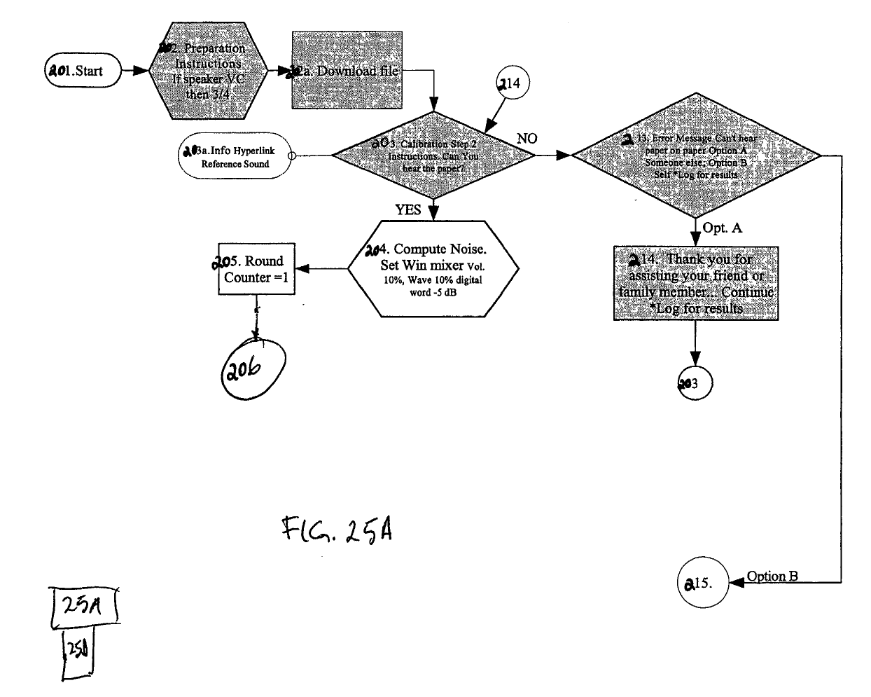

When the process is done, the web page of FIG. 24 is presented which acts as a closing presentation for the process.

The interactive presentation shown in FIGS. 11 through 24 is adapted for presentation using a full function browser in a personal computer with a large format display, and coupled to the Internet. In other types of consumer devices, such as mobile phones or personal digital assistants, the presentation is adapted to the format of the display available. Also, the types of software components that are downloaded from the server to the remote device to support the hearing test are adapted to be architecture of the platforms used during the testing process.

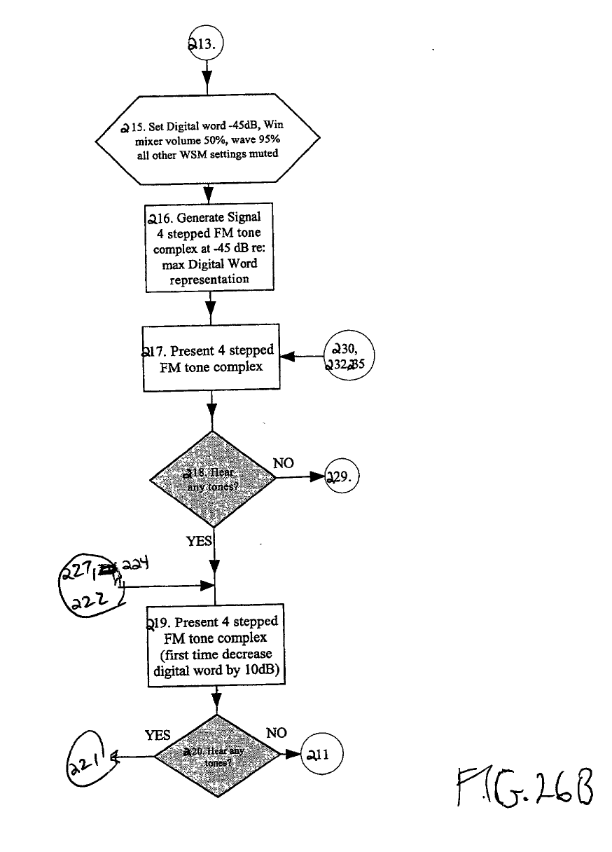

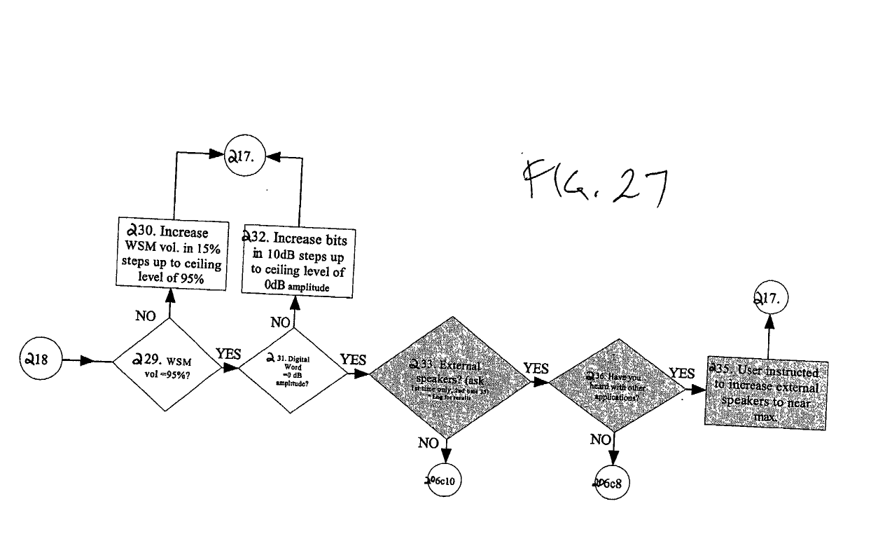

One embodiment of a main flow for a calibration process according to the present invention is shown in FIGS. 25A-25B. The process begins with a start signal (block 201). Next, environment preparation instructions are displayed, such a shown in FIG. 13, instructing the user to adjust the volume control on external speakers to about ¾ of maximum, or other level selected to provide a reasonable operating range for the calibration process (block 202). Next, a file is downloaded from the server to the electronic device carrying components of the calibration program. The calibration program provides instructions such as shown in FIGS. 14 and 15 to the user about how to produce a calibration sound. In a next step, the program determines whether the user can hear the calibration sound (block 203). If at block 203, the user indicates that the calibration sound cannot be heard, then the procedure branches to block 213, where the user is offered an option to find someone to assist him or her to calibrate the system, or to perform an alternative calibration process. If the user selects the alternate calibration process, then the program branches to block 215 of FIG. 26B. If the user selects the alternative in which a friend assists with calibration, then a thank you screen is presented (block 214), and the algorithm branches to block 203. Block 203a indicates a hyperlink to an informational page about the calibration sound. If at block 203, the user indicates that the calibration sound can be heard, then the process computes a noise signal, and sets the programmable volume control parameters, including the Master volume and wave out controls in a Windows mixer panel for example, for the audio resources on the electronic device. The digital word for production of the noise is set at a initial amplitude value of, for example, about −5 dB (block 204). Next, a round counter is set for round 1 (block 205).

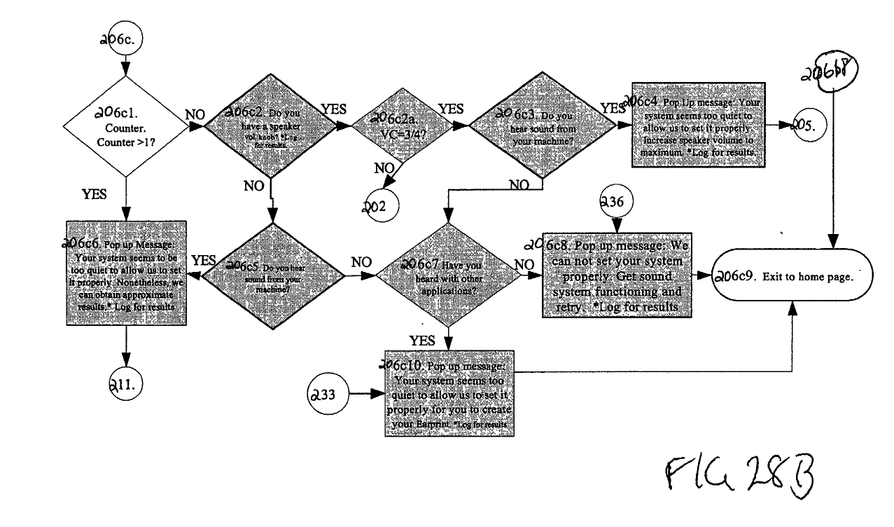

At this point, the algorithm proceeds to the test page, such as shown in FIG. 18. During display of the test page, the computed noise is played on command from the test subject at the current settings of the audio resources. A user input is provided by selecting the up arrow or the down arrow. Selecting the up arrow increases both the volume and wave out parameters in 5 percent steps to a maximum of 95 percent setting. The down arrow decreases both the volume and wave out settings in 5 percent steps, to a minimum of 0 percent (Block 206). If the test succeeds at a digital word setting of between 5 percent and 95 percent, then the sound is stopped in the process proceeds to step 207. However, if the user selects three down arrows in a row at the 0 percent setting, or indicates a match at a level of less than 5 percent, then the sound is stopped, and it is presumed that the user has a system that operates loudly. In this condition, the process branches to an error flow at beginning at step 206b5 of FIG. 28A. Likewise, if the user executes the click on three up arrows in a row at a setting of 95 percent or indicates a match at a level of about 95 percent, then the sound is stopped, the user is presumed to have a quiet system. In this condition, the algorithm branches to an error flow beginning at block 206c1 of FIG. 28B.

With a successful test completion at block 206a, the process proceeds to block 207, in which the round counter is tested. In one example, the round counter is tested for match with the number (4 in this example) indicating that three rounds have been completed. In an alternative test, the round counter is compared with a value of three indicating that 2 rounds have been completed. The number of rounds chosen is a trade-off between the accuracy of the test, and the time taken to complete the test. If the round counter has not reached its final value, then the round counter is incremented (block 208), and the mixer values are set to near ½ of the value determined by the process of block 206a, rounded to the closest 5 percent, with a minimum setting of around 10 percent for both of the programmable parameters (block 209). Then the algorithm branches back to block 206 to repeat the test.

If at block 207, it is determined that the round counter has reached its final value, then the process determines whether the user has been executing a calibration process with an assistant, according to the option provided at block 213 (block 210). If an assistant was used, then the assistant is instructed to allow the test subject to do the test (block 210b). After step 210, if it is not an assisted process, or after step 210b, the process proceeds to present a practice hearing assessment test, using the current mixer settings. If the practice test is entered from steps 210 or 210b, the practice uses a digital word setting of minus 5 dB for the practice tones. If in this block, the practice test is entered from step 220 of the alternate calibration flow, then the digital word is set a level 30 dB above the final settings of the calibration process, using a 20 percent mixer volume setting. The practice test times out after 60 seconds, for example, and presents the user choices to continue with an actual hearing assessment test, to continue with practicing, or to return to instructions (block 211). Blocks 211a and 211b indicate hyperlinks to informational pages describing features of the test process, including the “guessing” involved, and allowing the user to hear the tones to be used. If the user proceeds, then the process creates an ear print according to hearing assessment test flow, such as described above (block 212).

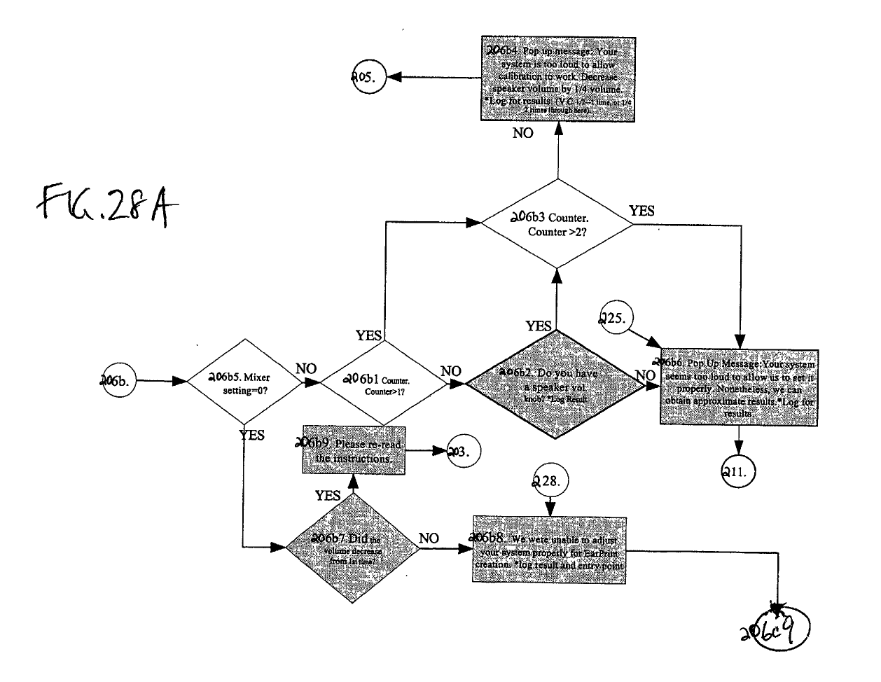

FIGS. 26A and 26B illustrate the alternate calibration flow entered in FIG. 26B from block 213 of FIG. 25B. The alternate flow is entered if the test subject indicates that the calibration sound cannot be heard. In this alternative, a “relative” hearing loss says is performed which allows for an estimated calibration that is quick, but has little inherent accuracy. In a first step, the digital word for controlling the audio resources is set to about −45 dB, the Windows Master volume and wave out settings are set at 50 percent and 95 percent respectively, and the other Windows mixer settings are muted (block 215). Next, the process generates the signals to produce 4 stepped FM tones at the minus 45 dB setting (block 216). Preferably, the tones are slightly worbled. Then the tone complex is presented at the electronic device (block 217). The user is prompted to indicate whether any of the four tones were heard (block 218). If no tones were heard, then the process proceeds to the error flow beginning in block 229 in FIG. 27. If the user indicates that the tones were heard, the tone complex is presented again, with the digital word setting decreased by 10 dB (block 219). The user is prompted again to indicate whether any tones were heard (block 220). If no tones were heard, then the process branches to step 211 in the flow of FIG. 25B to proceed with the hearing assessment test. If any tones are heard at step 220, then the process determines whether the digital word has been decremented to a value of −65 dB (block 221). If at step 221, the digital word had not been decremented to the minimum setting, then the process decreases the digital word in 10 dB steps (block 222), and returns to block 219 to present the tone complex again. If at step 221, the digital word value had been decremented to its minimum setting for this test flow, the process proceeds to determine if the wave out setting for the Windows mixer is set at 10 percent (block 223). If the wave out setting has not been decreased to 10 percent, then it is decreased in 10 percent steps, and the process returns to step 219 to present the tone complex with a new settings (block 224). If at step 223, the wave out setting had been reduced to its minimum for this test, then the user is prompted with the screen asking whether the sound has been actually getting quieter, to determine whether or not the process is actually adjusting the audio output (block 228), unless the step 228 is entered a second time during the calibration flow. If at step 228, the user indicates that the sounds are not getting quieter, then the algorithm branches to the error flow that begins at block 206b8 of FIG. 28A. If at step 228, it is determined that the sound has been getting quieter, then the process prompts the user to determine whether the electronic device has external speakers, on the first time through (block 225). If the user does not have external speakers, then the process branches to the error flow that begins at block 206b6 in FIG. 28A. If at block 225, the user indicates that external speakers are coupled with electronic device, then the process determines whether the external speakers have been set to their lowest level for this test, for example ¼. If the speakers have not been set to the lowest level for the test, then the process returns instructs the user to reduce the external speakers volume by ¼ (block 227) and returns to present the tone complex at the new settings in block 219. If at block 226, it is determined that the speakers have been set to their lowest level for this test, the process branches to the error flow that begins at block 206b8 in FIG. 28A.

Successfully executing the alternate calibration flow shown in FIGS. 26A and 26B results in determining a setting at which the user does not hear any tones in the four tone complex. At this setting, a hearing assessment can be made beginning at a level which is significantly higher, such as 30 dB higher one example, that has little inherent accuracy, but may otherwise provide a rough estimate of the hearing profile they could be used for some purposes.

The error flow entered from block 218 of FIG. 26B is shown in FIG. 27. In the first step, the process determines whether the Windows Master volume setting has reached 95 percent (block 229). If the Master volume setting has not reached the ceiling level of 95 percent, then it is increased in 15 percent steps (block 230), and the process returns to block 217 of FIG. 26B. If at block 229, it is determined that the Master volume setting has been stepped up to the ceiling level, then it is determined whether the digital word used to signal the sound is set at 0 dB amplitude (block 231). If the digital word is not at 0 dB, then it is increased in 10 dB steps up to a ceiling level of 0 dB amplitude (block 232), and the process returns to present the tone complex at step 217 in FIG. 26B. If at step 231, it is determined that the digital word has reached the ceiling level, then the user is prompted in the first time through the flow to indicate whether external speakers are being used (block 233). If external speakers are not being used, then the error flow which begins at block 206c10 in FIG. 28B is executed. If at step 233, it is determined that external speakers are being used, then the user is prompted to indicate whether sounds have been heard with other applications (block 236). If the sounds have not been heard with other applications, then the error flow beginning at step 206c 8 of FIG. 28B is executed. If at step 236, it is determined that the user has heard sounds with other applications, or on the second time through the flow at block 233, then the user is instructed to increase the external speaker volume to near their maximum setting (block 235), and process proceeds to block 217 of FIG. 26B.

FIGS. 28A and 28B illustrate the error flows which are entered if the user has a loud system or a quiet system, respectively. FIG. 28A is entered primarily from the block 206b of FIG. 25B. The process first determines whether the mixer volume and wave settings have reached 0 (block 206b5). If not, the process determines whether the round counter is greater than 1 (block 206b1). If the round counter is not greater than 1, then the user is prompted to find out whether a speaker volume control is available (block 206b2). If the user does not have an available volume control, then the system provides a message which indicates to the user that the system seems too loud to be set properly, and suggests that approximate hearing assessment results might be obtained (block 206b6). From step 206b6, the process proceeds to the step 211 of FIG. 25B. If at step 206b1, it is determined that the round counter is greater than 1, or in step 206b2 it is determined that the user has a volume control knob, then the process determines whether the round counter is greater than 2, for the example in which three rounds are executed. If the round counter is not greater than 2, then the system provides a prompt to the user indicating that the system is too loud for calibration to work, and instructing user to decrease the volume control by ¼ volume (block 206b4). After step 206b4, the process loops back to step 205 of FIG. 25A. If at step 206b3, the round counter is greater than 2, the process loops to block 206b6 which operates as described above.

If at step 206b5, it is determined that the mixer setting for the volume control and wave out have reached the minimum setting, the user is prompted to determine whether the perceived volume decreased during the flow (block 206b7). If the volume did decrease, then the user is prompted to re-read the instructions (block 206b9), and then the process loops to block 203 to attempt the calibration process again. If at block 206b7, the user indicates that the volume did not decrease during the flow, then the user is prompted with the screen that indicates that the test was unable to adjust the system properly for the ear print creation (block 206b8), and the process returns to the homepage.

FIG. 26B is entered from block 206c of FIG. 25B, in the event that the test flow suggests that the user's system is too quiet. First, it is determined whether the round counter is greater than 1 (block 206c1). If the round counter is greater than 1, then the user is presented a message indicating that the system seems to be too quiet for properly executing the test, but suggests that approximate results might be obtained, and returns the user to block 211 of FIG. 25B. If at block 206c1, the round counter is not greater than 1, then the user is prompted to find out whether a speaker volume control is available (block 206c2). If the speaker volume control is available, the process determines whether the volume control is set to ¾ level (block 206c2a). If the volume control is not set to ¾ level, the process returns to step 202 of FIG. 25A. If it is determined that the volume control is set to ¾ level, then the user is prompted to find out whether any sound was heard from the machine (block 206c3). If a sound was heard, then the user is prompted with a message that states that the system seems too quiet to allow proper setting, and suggests that the user increase the speaker volume to the maximum (block 206c4). Then the process returns to step 205 of FIG. 25A.

If at step 206c2, no volume control is available to the user, then the user is prompted to find out whether any sound was heard from the machine (block 206c5). If a sound was heard, then the process returns to step 206c6 described above. If no sound was heard at step 206c5, or if no sound was heard from step 206c3, the process prompts the user to determine whether sound had been heard with other applications (block 206c7). If sounds have been heard, then the user is presented a screen that suggests the system is too quiet to allow for proper setting, and for obtaining an ear print (block 206c10), and the process returns to the homepage (block 206c9). If at block 206c7, the user indicates that no sound had been heard with other applications, then a message is presented telling the user that the system cannot be set properly, and requesting that user retry after the sound system is properly functioning on the electronic device (block 206c8). Finally, that system returns to the homepage at block 206c9.

The present invention allows for that a reasonably accurate evaluation of the hearing capability of a subject to be accessed remotely using a computer system. Calibration of the electronic device used remotely for the hearing testing is an imperative for achieving useful results. The present invention provides such calibration, without requiring special equipment to be delivered to the remote site. Thus, remote, self-administered hearing tests are enabled, by enabling operators to calibrate their systems in an manner that is reasonable from a cost and convenience perspective, while accurate enough to generate usable results.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the appended claims.

What is claimed is:

1. A method for calibrating audio resources on a consumer electronic device, comprising:
prompting a user of the device to make a calibration sound using an item likely to be available to the user, other than the audio resources on the device;
executing a process using audio resources in the device to calibrate the device with reference to the calibration sound.

2. The method of claim 1, wherein said process includes determining a setting of the audio resources which results in production of a masking sound by the device which masks the calibration sound; and saving the setting.

3. The method of claim 1, wherein said device comprises a computer including a programmable volume control parameter for the audio resources, said process includes determining a setting of the audio resources which results in production of a masking sound by the device which masks the calibration sound, and said setting comprises a setting for said programmable volume control parameter.

4. The method of claim 1, wherein said process includes determining a setting of the audio resources which results in production of a masking sound by the device which masks the calibration sound; and including
instructing the user to signal completion of said process when a condition is achieved in which the masking sound masks the calibration sound, wherein said instructions use terminology semantically equivalent to “drowns out” to describe the condition.

5. The method of claim 1, including generating a control signal to produce an audio stimulus on the device, the control signal being based upon said calibration.

6. The method of claim 1, including generating a control signal to produce an audio stimulus on the device, the control signal being based upon said calibration, and resulting in said audio stimulus having a sound pressure level within 10 dB of a predicted level.

7. The method of claim 1, wherein said device includes a display, and said prompting includes displaying instructions to the user.

8. The method of claim 1, wherein said device includes a display, and said prompting includes displaying instructions to the user, the instructions including a description of a technique for making the calibration sound using the item, and a description of a process for controlling the device to generate a masking sound.

9. The method of claim 1, wherein said item comprises a sheet of paper.

10. The method of claim 1, wherein said item comprises a sheet of paper, and said device includes a display, and said prompting includes displaying instructions to the user, the instructions including a description of a technique for making the calibration sound by rubbing the sheet of paper, and a description of a process for controlling the device to generate the masking sound.

11. The method of claim 1, wherein said item comprises two sheets of paper, said prompting includes providing instructions to the user, the instructions including a description of a technique for making the calibration sound by folding a first piece of paper, laying the second piece of paper on a desk, and rubbing the first sheet of paper on the second sheet of paper, and a description of a process for controlling the device to generate the masking sound.

12. The method of claim 1, wherein said item comprises an alphanumeric keyboard, said prompting includes providing instructions to the user, the instructions including a description of a technique for making the calibration sound by striking the keyboard.

13. The method of claim 1, wherein said item comprises a pencil, said prompting includes providing instructions to the user, the instructions including a description of a technique for making the calibration sound by rubbing the pencil on a surface.

14. The method of claim 1, wherein said item comprises a desk, said prompting includes providing instructions to the user, the instructions including a description of a technique for making the calibration sound by tapping the desk.

15. The method of claim 1, wherein said item comprises a coin, said prompting includes providing instructions to the user, the instructions including a description of a technique for making the calibration sound by dropping the coin on a surface.

16. The method of claim 1,wherein said item comprises a keychain, said prompting includes providing instructions to the user, the instructions including a description of a technique for making the calibration sound by jingling the keychain.

17. The method of claim 1, wherein said process comprises an interaction in which the user supplies input signals to adjust a masking sound until a condition is met.

18. The method of claim 1, wherein said process comprises a routine which automatically adjusts a masking sound, and an interaction in which the user supplies an input signal to indicate that a condition is met.

19. The method of claim 1, including determining if the user can hear the calibration sound, and if not, then executing an alternative process.

20. The method of claim 1, including determining if the user can hear the calibration sound, and if not, then executing an alternative process, the alternative process determining a setting at which the user cannot hear a set of test sounds.

21. The method of claim 1, wherein said process includes at least one flow which is executed if an error condition occurs, that prompts the user to perform an act to correct a possible source of the error.

22. The method of claim 1, wherein said process includes using the audio resources to produce a masking sound by the device, and determining a setting for the audio resources at which the masking sound masks the calibration sound.

23. The method of claim 1, wherein said process includes providing input to the audio resources to produce a masking sound by the device, the input including substantially randomly generated signals, and determining a setting for the audio resources at which the masking sound masks the calibration sound.

24. The method of claim 1, wherein said process includes providing input to the audio resources to produce a masking sound by the device, the input including signals to produce a masking sound having an audio spectrum matching a spectrum expected for the calibration sound, and determining a setting for the audio resources at which the masking sound masks the calibration sound.

25. A method for calibrating audio resources on a consumer electronic device, comprising:
prompting a user of the device to make a calibration sound using an item other than the audio resources on the device; and
executing a process using audio resources in the device to calibrate the device with reference to the calibration sound, wherein said process includes determining a setting of the audio resources which results in production of a masking sound by the device which masks the calibration sound.

26. The method of claim 25, wherein said prompting includes providing instructions to the user describing said process, including instructing the user to signal completion of said process when a condition is achieved in which the masking sound masks the calibration sound, wherein said instructions use terminology semantically equivalent to “drowns out” to describe the condition.

27. The method of claim 25, including saving the setting.

28. The method of claim 25, including generating a control signal to produce an audio stimulus on the device, the control signal being based upon said setting.

29. The method of claim 25, including generating a control signal to produce an audio stimulus on the device, the control signal being based upon said setting, and resulting in said audio stimulus having a sound pressure level within about 10 dB of a predicted level.

30. The method of claim 25, wherein said device includes a display, and said prompting includes displaying instructions to the user.

31. The method of claim 25, wherein said device includes a display, and said instructions including a description of a technique for making the calibration sound, and a description of a process for controlling the device to generate the masking sound.

32. The method of claim 25, wherein said device comprises a computer including a programmable volume control parameter for the audio resources, and said setting comprises a setting for said programmable volume control parameter.

33. The method of claim 25, wherein said process comprises an interaction in which the user supplies input signals to adjust the masking sound until the condition is met.

34. The method of claim 25, wherein said process comprises a routine which automatically adjusts the masking sound, and an interaction in which the user supplies an input signal to indicate that the condition is met.

35. The method of claim 25, including determining if the user can hear the calibration sound, and if not, then executing an alternative process.

36. The method of claim 25, including determining if the user can hear the calibration sound, and if not, then executing an alternative process, the alternative process determining a setting at which the user cannot hear a set of test sounds.

37. The method of claim 25, wherein said process includes at least one flow which is executed if an error condition occurs, that prompts the user to perform an act to correct a possible source of the error.

38. The method of claim 25, wherein said process includes providing input to the audio resources to produce the masking sound, the input including substantially randomly generated signals.

39. The method of claim 25, wherein said process includes providing input to the audio resources to produce the masking sound, the input including signals to produce a masking sound having an audio spectrum matching a spectrum expected for the calibration sound.

40. A method for calibrating audio resources on a consumer electronic device, comprising:
prompting a user of the device to make a calibration sound using an item other than the audio resources on the device; and
executing a process using audio resources in the device to calibrate the device with reference to the calibration sound, wherein said process includes determining a setting of the audio resources which results in production of a test sound by the device, the masking sound having an audio spectrum matching a spectrum expected for the calibration sound..

41. The method of claim 40, wherein said prompting includes providing instructions to the user describing said process, including instructing the user to signal completion of said process when a condition is achieved in which the test sound masks the calibration sound, wherein said instructions use terminology semantically equivalent to “drowns out” to describe the condition.

42. The method of claim 40, including generating a control signal to produce an audio stimulus on the device, the control signal being based upon said setting.

43. The method of claim 40, including generating a control signal to produce an audio stimulus on the device, the control signal being based upon said setting, and resulting in said audio stimulus having a sound pressure level within about 10 dB of a predicted level.

44. The method of claim 40, wherein said device includes a display, and said prompting includes displaying instructions to the user.

45. The method of claim 40, wherein said device includes a display, and said prompting includes displaying a description of a technique for making the calibration sound, and a description of a process for controlling the device to generate the test sound.

46. The method of claim 40, wherein said device comprises a computer including a programmable volume control parameter for the audio resources, and said setting comprises a setting for said programmable volume control parameter.

47. The method of claim 40, wherein said process comprises an interaction in which the user supplies input signals to adjust the test sound until the condition is met.

48. The method of claim 40, wherein said process comprises a routine which automatically adjusts the test sound, and an interaction in which the user supplies an input signal to indicate that the condition is met.

49. A method for conducting a hearing test using a computer program, comprising:
establishing a communication channel between a remote device and a server in a communication network;
prompting a user of the remote device, using resources provided via said communication channel, to make a calibration sound using an item likely to be available to the user, other than the audio resources on the device;
executing a calibration process using audio resources in the device to determine a calibration of the audio resources which results in production of a masking sound by the remote device which masks the calibration sound; and
executing a computer program to present a hearing test to the user of the remote device, wherein the computer program comprises a routine, responsive to said calibration.

50. The method of claim 49, wherein said computer program adaptively selects stimuli for the hearing test based upon said interaction produced at the remote device using the calibration.

51. The method of claim 49, wherein said hearing test comprises an N-alternative forced choice interaction.

52. The method of claim 49, wherein the communication network comprises a packet switched network.

53. The method of claim 49, wherein the communication network comprises a network executing according a standard internet protocol.

54. The method of claim 49, wherein the channel comprises a connection according to a standard transmission control protocol over a standard internet protocol (TCP/IP).

55. The method of claim 49, wherein the channel comprises a link through a cellular telephone network.

56. The method of claim 49, wherein the channel comprises a link through a pager network.

57. The method of claim 49, wherein the remote device comprises one of a mobile phone, a home computer, and a hand held computing platform.

58. The method of claim 49, wherein said calibration process includes determining a setting of the audio resources which results in production of the masking sound by the device which masks the calibration sound; and saving the setting.

59. The method of claim 49, wherein said device comprises a computer including a programmable volume control parameter for the audio resources, said calibration process includes determining a setting of the audio resources which results in production of the masking sound by the device which masks the calibration sound, and said setting comprises a setting for said programmable volume control parameter.

60. The method of claim 49, wherein said calibration process includes instructing the user to signal completion of said calibration process when a condition is achieved in which the masking sound masks the calibration sound, wherein said instructions use terminology semantically equivalent to “drowns out” to describe the condition.

61. The method of claim 49, wherein the routine responsive to the calibration includes generating a control signal to produce an audio stimulus on the device, the control signal being based upon said calibration, and resulting in said audio stimulus having a sound pressure level within about 10 dB of a predicted level.

62. The method of claim 49, wherein said device includes a display, and said prompting includes displaying instructions to the user.

63. The method of claim 49, wherein said device includes a display, and said prompting includes displaying instructions to the user, the instructions including a description of a technique for making the calibration sound using the item, and a description of a process for controlling the device to generate the masking sound.

64. The method of claim 49, wherein said item comprises a sheet of paper.

65. The method of claim 49, wherein said item comprises a sheet of paper, and said device includes a display, and said prompting includes displaying instructions to the user, the instructions including a description of a technique for making the calibration sound by rubbing the sheet of paper, and a description of a process for controlling the device to generate the masking sound.

66. The method of claim 49, wherein said item comprises two sheets of paper, said prompting includes providing instructions to the user, the instructions including a description of a technique for making the calibration sound by folding a first piece of paper, laying the second piece of paper on a desk, and rubbing the first sheet of paper on the second sheet of paper, and a description of a process for controlling the device to generate the masking sound.

67. The method of claim 49, wherein said item comprises an alphanumeric keyboard, said prompting includes providing instructions to the user, the instructions including a description of a technique for making the calibration sound by striking the keyboard.

68. The method of claim 49, wherein said item comprises a pencil, said prompting includes providing instructions to the user, the instructions including a description of a technique for making the calibration sound by rubbing the pencil on a surface.

69. The method of claim 49, wherein said item comprises a desk, said prompting includes providing instructions to the user, the instructions including a description of a technique for making the calibration sound by tapping the desk.

70. The method of claim 49, wherein said item comprises a coin, said prompting includes providing instructions to the user, the instructions including a description of a technique for making the calibration sound by dropping the coin on a surface.

71. The method of claim 49, wherein said item comprises a keychain, said prompting includes providing instructions to the user, the instructions including a description of a technique for making the calibration sound by jingling the keychain.

72. The method of claim 49, wherein said calibration process comprises an interaction in which the user supplies input signals to adjust the masking sound until the condition is met.

73. The method of claim 49, wherein said calibration process comprises a routine which automatically adjusts the masking sound, and an interaction in which the user supplies an input signal to indicate that the condition is met.

74. The method of claim 49, including determining if the user can hear the calibration sound, and if not, then executing an alternative calibration process.

75. The method of claim 49, including determining if the user can hear the calibration sound, and if not, then executing an alternative calibration process, the alternative calibration process determining a setting at which the user can hear a set of test sounds.

76. The method of claim 49, wherein said calibration process includes at least one flow which is executed if an error condition occurs, that prompts the user to perform an act to correct a possible source of the error.

77. The method of claim 49, wherein said calibration process includes determining a setting for the audio resources at which the masking sound masks the calibration sound.

78. The method of claim 49, wherein said calibration process includes providing input to the audio resources to produce the masking sound, the input including substantially randomly generated signals, and determining a setting for the audio resources at which the masking sound masks the calibration sound.

79. The method of claim 49, wherein said calibration process includes providing input to the audio resources to produce the masking sound, the input including signals to produce the masking sound having an audio spectrum matching a spectrum expected for the calibration sound, and determining a setting for the audio resources at which the masking sound masks the calibration sound.

80. An apparatus comprising:
a data processor which executes instructions;
a communication interface coupled to the processor; and
memory coupled to the data processor which stores instructions in a form readable by the data processor, the instructions specifying processes which
establish a communication channel to a remote device across the communication interface;
prompt a user of the remote device, using resources provided via said communication channel, to make a calibration sound, using something other than the audio resources on the device;
execute a calibration process using audio resources in the device to determine a calibration of the audio resources which results in production of a masking sound by the remote device which masks the calibration sound; and
execute a computer program to present a hearing test to the user of the remote device, wherein the computer program comprises a routine, responsive to said calibration.

81. The apparatus of claim 80, wherein said computer program adaptively selects stimuli for the hearing test based upon said interaction produced at the remote device using the calibration.

82. The apparatus of claim 80, wherein said hearing test comprises an N-alternative forced choice interaction.

83. The apparatus of claim 80, wherein the communication interface is coupled to a cellular telephone network.

84. The apparatus of claim 80, wherein the communication interface is coupled to a pager network.

85. The apparatus of claim 80, wherein the remote device comprises one of a mobile phone, a home computer, and a hand held computing platform.

86. The apparatus of claim 80, wherein said remote device comprises a computer including a programmable volume control parameter for the audio resources, said calibration process includes determining a setting of the audio resources which results in production of the masking sound by the device which masks the calibration sound, and said setting comprises a setting for said programmable volume control parameter.

87. The apparatus of claim 80, wherein said calibration process includes determining a setting of the audio resources which results in production of the masking sound by the device which masks the calibration sound; and including
the instructions further specify a process which instructs the user to signal completion of said calibration process when the condition met, the condition being the perception the masking sound masks the calibration sound, wherein said instructions use terminology semantically equivalent to “drowns out” to describe the condition.

88. The apparatus of claim 80, wherein said hearing test includes generating a control signal to produce an audio stimulus on the device, the control signal being based upon said calibration, and resulting in said audio stimulus having a sound pressure level within 10 dB of a predicted level.

89. The apparatus of claim 80, wherein said remote device includes a display, and said prompting includes displaying instructions to the user on said display.

90. The apparatus of claim 80, wherein said remote device includes a display, and said prompting includes displaying instructions to the user on said display, the instructions including a description of a technique for making the calibration sound using an item likely to be available to the user, and a description of a process for controlling the device to generate a masking sound.

91. The apparatus of claim 90, wherein said item comprises a sheet of paper.

92. The apparatus of claim 90, wherein said item comprises a sheet of paper, and said device includes a display, and said prompting includes displaying instructions to the user, the instructions including a description of a technique for making the calibration sound by rubbing the sheet of paper, and a description of a process for controlling the device to generate the masking sound.

93. The apparatus of claim 90, wherein said item comprises two sheets of paper, said prompting includes providing instructions to the user, the instructions including a description of a technique for making the calibration sound by folding a first piece of paper, laying the second piece of paper on a desk, and rubbing the first sheet of paper on the second sheet of paper, and a description of a process for controlling the device to generate the masking sound.

94. The apparatus of claim 90, wherein said item comprises an alphanumeric keyboard, said prompting includes providing instructions to the user, the instructions including a description of a technique for making the calibration sound by striking the keyboard.

95. The apparatus of claim 90, wherein said item comprises a pencil, said prompting includes providing instructions to the user, the instructions including a description of a technique for making the calibration sound by rubbing the pencil on a surface.

96. The apparatus of claim 90, wherein said item comprises a desk, said prompting includes providing instructions to the user, the instructions including a description of a technique for making the calibration sound by tapping the desk.

97. The apparatus of claim 90, wherein said item comprises a coin, said prompting includes providing instructions to the user, the instructions including a description of a technique for making the calibration sound by dropping the coin on a surface.

98. The apparatus of claim 90, wherein said item comprises a key chain, said prompting includes providing instructions to the user, the instructions including a description of a technique for making the calibration sound by jingling the key chain.

99. The apparatus of claim 80, wherein said calibration process comprises a routine which automatically adjusts a masking sound, and an interaction in which the user supplies an input signal to indicate that the condition is met.

100. The apparatus of claim 80, wherein said calibration process comprises a routine which determines if the user can hear the calibration sound, and if not, then executes an alternative calibration process.

101. The apparatus of claim 80, wherein said calibration process comprises a routine which determines if the user can hear the calibration sound, and if not, then executes an alternative calibration process, the alternative calibration process determining a setting at which the user cannot hear a set of test sounds.

102. The apparatus of claim 80, wherein said calibration process includes at least one flow which is executed if an error condition occurs, that prompts the user to perform an act to correct a possible source of the error.

103. The apparatus of claim 80, wherein said calibration process includes using the audio resources to produce the m asking sound, and determines a setting for the audio resources at which the masking sound masks the calibration sound.

104. The apparatus of claim 80, wherein said calibration process includes providing input to the audio resources to produce the masking sound, the input including substantially randomly generated signals, and determines a setting for the audio resources at which the masking sound masks the calibration sound.

105. The apparatus of claim 80, wherein said calibration process includes providing input to the audio resources to produce the masking sound, the input including signals to produce a masking sound having an audio spectrum matching a spectrum expected for the calibration sound, and determines a setting for the audio resources at which the masking sound masks the calibration sound.
